(19) United States
(12) Patent Application Publication
(10) Pub. No.: US 2026/0193627 A1
(43) Pub. Date: Jul. 9, 2026

(54) IMMOBILISED LIPASE AND METHOD OF PRODUCING BIODIESEL USING THE SAME

(71) Applicant: VAAL UNIVERSITY OF TECHNOLOGY, Johannesburg (ZA)

(72) Inventors: Naser Aliye FETO, Johannesburg (ZA); Makwena Justice MOLOTO, Johannesburg (ZA); Mpho Gladys NKHI, Johannesburg (ZA)

(21) Appl. No.: 19/134,281

(22) PCT Filed: Jan. 5, 2023
(86) PCT No.: PCT/ZA2023/050002

§ 371 Date: May 30, 2025

(30) Foreign Application Priority Data
Nov. 30, 2022 (ZA) 2022/13010

(51) Int. Cl. C12N 9/20; C10L 1/02; C12N 11/14; C12P 7/649

(52) U.S. Cl. CPC C12N 9/20; C10L 1/02; C12N 11/14; C12P 7/649; C10L 2200/0476; C10L 2200/0484; C10L 2290/26

(57) ABSTRACT

The invention relates to a lipase isolated from the goat gut rumen metagenome having protein SEQ ID 1 of 298 amino acids and being encoded for by gene SEQ ID1. The invention relates more specifically to this lipase being engineered to be an immobilised lipase, the same being immobilised on Polyethylene glycol (PEG) coated iron Magnetic Nanoparticles (MNPs). The immobilised lipase is indicated for use in a method of converting a waste oil to biodiesel, the method comprising utilizing the immobilised lipase to catalyse the conversion of said waste oils to biodiesel in the presence of a short-chain alcohol. The triacylglycerol in the waste oils being converted to alkyl esters (biodiesel) and glycerin in a reaction with an alcohol which is catalysed by said lipases.

### FIELD OF THE INVENTION

The invention relates to a biodiesel production using enzyme-based methodology. More particularly, the invention relates to the enzyme used in this method, said enzyme being a lipase. In particular, the enzyme is an immobilised lipase from the goat gut rumen metagenome.

### BACKGROUND TO THE INVENTION

Biodiesel is a form of diesel fuel, derived from plants, animals, or oil-based waste products. It is an incredibly important fuel source from an economic point of view given that the global production of the same in 2022 reached approximately 53 million tons, with Indonesia being the largest producer of the same. Waste oils are an attractive source of feedstock for use in the production of biodiesel as they have a high free fatty acid content which is easily convertible to biodiesel. For example, in South Africa approximately 28 million tons of waste oil (predominantly waste vegetable oil) is produced annually but only two to three million of this is converted to biodiesel by one of the five large-scale biodiesel producers in the country utilizing chemical rather than enzyme-based processes. Therefore, there is a large potential for growth in the South African market alone and hence a variety of processes to produce the same should be considered. Even though the South African market is utilised as an illustrative example of the commercial potential for the invention the same is in no manner limited to this market and is applicable within a large worldwide market which has grown fifteen percent more than projected during 2021 to 2022 period.

Also known as “green-fuel” biodiesel is a low environmental impact fuel allowing for a reduction in carbon monoxide production upon combustion of the same by up to eighty percent as compared to fossil fuels. In addition, the carbon dioxide produced by the combustion thereof can be absorbed by plants within one year. The greenhouse mitigation potential of biofuels is favorable, in some instances resulting in negative emissions and in less favorable scenarios emission levels comparable to fossil fuels. However, even where emission levels are comparable to those of fossil fuels biodiesel still shows significant advantages given the lower costs and shorter time required when producing the same.

The other advantage of biodiesel is that it is a so-called drop-in fuel which means it is produced over a shorter period than fossil fuels which are produced through a slow natural process. This is especially ideal given that there is a limited amount of fossil fuel available which necessitates the development of alternative manners of accessing fuel sources. In most industrial and commercial applications, it is possible to use biodiesel instead of fossil fuel. For example, biodiesel is capable of being used in diesel engines without modification and of being mixed with varying ratios of petroleum when intended to be used in petroleum engines.

Biodiesel is produced using one of two methods, either via chemical conversion or enzymatic conversion: the catalysts in each instance being chemical or enzyme-based.

The chemical process uses relatively pure (“clean”) lipid feedstock, such as animal fat (tallow), yellow grease, lard, soybean oil or vegetable oil is used. The feedstock contains triglycerides (triacylglycerol) which are the main constituents of body fat in animals and in vegetable fat. The triglycerides are chemically reacted with an alcohol, producing a mixture of fatty acid esters (biodiesel) and a glycerol byproduct by the process of transesterification. Fatty Acid Methyl Ester (FAME) is the most utilised chemical process to produce biodiesel where methanol (converted to sodium methoxide) is used to produce methyl esters. Fatty Acid Ethyl Ester (FAEE) is a similar process wherein ethanol is used instead of methanol. There are some advantages to the use of FAEE rather than FAME in that, ethanol is more environmentally friendly and more renewable than methanol. However, ethanol is less reactive and more expensive and hence the use of methanol is preferred.

The utilization of chemical catalysts in the production of biodiesel is not ideal as chemicals are harsh and not environmentally friendly. Furthermore, the use of a chemical process is costly as the feedstock needs to be pretreated (“cleaned”) prior to the conversion of the same to biodiesel. The cleaning of the feedstock and the methanol used in the conversion of the feedstock to biodiesel are costly and said costs are additionally increased by the need for energy inputs (like heating). In addition, the wastewater from the process needs to be treated to remove chemical discharge (which is toxic) adding further additional costs. The reaction is also not “reusable” as the chemical catalysts unlike immobilised-enzymatic catalysts cannot be recovered and reused in batch processes as explained hereunder. Glycerol is produced as a byproduct of this reaction yet unfortunately when this chemical process is used the same is of much lower grade quality than desired and hence the commercial potential of the same is severely limited.

It is possible to utilise a combination of chemical and enzymatic catalysts in the production of biodiesel in a process known as partial chemical transesterification. However, whilst this process attempts to solve the environmental impacts issues associated with the purely chemically catalysed processed partial chemical conversion processes (as shown for example in CA2595007) cannot fully address these concerns.

Given the abovementioned disadvantages associated with the chemically and partially chemically catalysed production of biodiesel enzyme-based catalysts are preferred as catalysts to be used in biodiesel production. Like all catalysts enzymes increase the reaction rate by lowering the activation energy. Enzymes are not consumed in said chemical reaction nor do they alter the equilibrium of the reaction. The specificity of an enzyme comes from the three-dimensional conformation of the same which allows it to bind to the substrate which is fundamental for catalysis. Enzyme catalysts are preferable as, not only is the use of an enzyme catalyst more environmentally friendly (as the enzyme is easily biodegraded), but it is also significantly less costly because of the reaction conditions required are milder in addition to no need for the pretreatment of feedstock nor subsequent wastewater treatment. Furthermore, biodiesel produced via enzymatic conversion is inexhaustible (unlike fossil fuels), renewable and non-toxic and results in the production of a fuel which is comparable in energy content to fossil fuels.

Lipases are usually the appropriate enzyme catalyst to produce biodiesel as their natural reaction is to hydrolyse the ester bond in the lipid. In this respect “lipase” is used as the general term for a group of enzymes that hydrolyse fats and which for this reason are utilised in wide variety of applications in the medical, chemical, medical and energy industries.

Most currently utilised enzyme-based methods are reliant on the use of free enzyme (non-immobilised) which results in enzyme inactivation during the reaction and the need for a new batch of enzymes every time a reaction is run which dramatically increases the costs associated with the production of biodiesel by this method. For this reason, methods have been developed which shown reliance on the use of immobilised enzymes as this allows for the control of enzyme concentration in a batch process and for the recovery of the same once the reaction is concluded for use in subsequent reactions. Enzymes may be immobilised via several different methods but are typically immobilised utilizing adsorption, entrapment, covalent coupling (conjugation), or cross-linking methods.

For lipases particularly, immobilisation results in increased catalytic activity which is ascribed to conformational changes in lipases that take place upon immobilisation. As regards the immobilisation of enzymes nanoparticles have shown promise given that they possess a high surface area to volume ratio and are a small size which allows for the avoidance of conformational restrictions. Several types of nanoparticles can be utilised, including but not limited to mesoporous silica, organic polymers and/or protein-coated microcrystals. However, for the purposes of the recovery of the immobilised enzymes magnetic nanoparticles have been found to be particularly useful as the same are easily recoverable from the reaction mixture.

The prior art in this field envisages a variety of catalysts and a variety of reaction mechanisms as further expanded upon hereunder:

WO2011104528 A2 envisages the use of immobilised lipases to produce the biodiesel compounds. However, it will be appreciated by a person skilled in the art that the processes described in this specification are vastly different to the process utilised to produce biodiesel as defined by the invention given that the use of interesterification is envisaged in this specification and this is not the reaction used in relation to this invention.

US2014/0075828A1 envisages the use of a lipase-catalysed transesterification process which is conducted in a supercritical carbon dioxide carrier to produce biodiesel. The feedstock utilised in this instance is the fat from lamb meat. Furthermore, this prior art envisages the use of an immobilised lipase enzyme wherein the enzyme is immobilised on an inert substrate. In this instance the lipase utilised is indicated specifically for the transesterification of raw animal fat which is distinct from the feedstock which is used for the purposes of this invention as will be appreciated by a person skilled in the art.

US20140017741 and various other prior art envisages the use of a fatty acid feedstock which is converted to biodiesel utilizing a two-step enzymatic reaction process. This specification envisages the use of an immobilised lipase extracted from a defined group of microbes. A person skilled in the art will appreciate that given that a defined group of microbes is utilised for the extraction and subsequent cloning of the lipases the resultant lipases that are available for use in the manufacture of biodiesel using this method will be limited to the specific group of lipases produced by said microbes. As such the group of lipases are specifically defined in this specification as is the feedstock which is distinct from the feedstock used for the purposes of the invention. As regards the use of specific lipases isolated from specified microbes the same is true with respect to the invention disclosed in US20100047884A1.

Given the analysis of the prior art there is a specific need for the provision of specific immobilised lipases which are suitable for the efficient and cost-effective use in the processing of waste oils to biodiesel.

### Definitions

For the avoidance of doubt the following terms will bear the meanings defined hereunder for the purposes of the interpretation of this specification:
Biodiesel means a fuel comprising mono-alkyl esters of long chain fatty acids derived from vegetable oils or animal fats;
Feedstock means the raw material utilised to supply an industrial process and in the case of biodiesel production may include, but not be limited to, raw animal fats, plant-based oils, and various waste oil products;
Inert means chemically inactive;
Interesterification means the process that rearranges the fatty acids of a fat product. In this process the ester bonds that connect the fatty acids to the glycerol hubs of the fat molecules are broken and reformed. These reactions may be catalysed using inorganic catalysts or enzymes;
Isoelectric point (pl) means the pH at which a protein (or other molecule), overall has a net zero charge;
Metagenome means the recovery and complete sequencing of genetic material extracted directly from an environmental sample, and this process of creating a metagenome is referred to as metagenomics;
Transesterification means the process of exchanging the organic group R″ of an ester with the organic group R′ of an alcohol. These reactions are often catalysed by the addition of an acid or base catalyst but can also be accomplished with the help of enzymes, particularly lipases. For the purposes of this invention transesterification is considered to be enzyme catalysed; Triglycerides means an ester derived from glycerol and three fatty acids.
Triglycerides are the main constituents of body fat in humans and other vertebrates, as well as vegetable fat. Triglycerides are also known as triacylglycerol and the same shall be used interchangeably.

### OBJECT OF THE INVENTION

It is an object of the invention to, at least in part, ameliorate some of the challenges associated with the currently utilized lipases and to the method of producing biodiesel using the same as currently applied in the art.

### SUMMARY OF THE INVENTION

The general field of the invention is related to lipases that are immobilised via covalent conjugation on to magnetic nanoparticles (MNP) and method of producing biodiesel using these lipases. Furthermore, the adsorbent MNP are recoverable which allows the immobilised enzymes to be utilised as catalysts for numerous reactions.

In accordance with a first aspect of the invention there is provided a lipase isolated from the goat gut rumen metagenome having protein SEQ ID 1.

There is also provided that protein SEQ ID1 comprises 298 amino acids.

There is also provided that protein SEQ ID 1 is encoded by for by a gene with SEQ ID 2.

There is also provided that the lipase has a molecular weight of about 33.4 KDa.

There is also provided that the lipase shows optimum catalysis at pH11 and 80° C. and has a pl of 5,713.

The preferred lipase was selected specifically for its stability at various temperatures and pHs and its specificity given that all of these factors influence the success of biodiesel production and commercial viability. Furthermore, it will be appreciated by a person skilled in the art that because the lipase is stable in alkaline conditions it may therefore be used together with an alkaline catalyst for the conversion of waste oils to biodiesel in a partial chemical transesterification reaction.

It will be appreciated by a person skilled in the art that albeit that the lipase envisaged in accordance with the first aspect of the invention is isolated from a natural source and therefore naturally occurring that the function of the same within its natural setting is not for the conversion of waste oils for biodiesel. Furthermore, with respect to the second aspect of the invention, which is described in more detail hereunder, the isolated lipase is not immobilised at the time of isolation and subsequent steps must be taken to immobilize the same. As such, an immobilised lipase which is applied specifically for the purposes of converting waste oils to biodiesel is neither a naturally occurring protein nor a naturally occurring application for the same.

In accordance with a second aspect of the invention there is provided an immobilised lipase having protein SEQ ID1, said immobilised lipase being immobilised on an adsorbent substrate.

There is also provided that protein SEQ ID1 is encoded for by gene SEQ ID 2 as set out in accordance with this first aspect of the invention.

There is also provided that the adsorbent substrate is a nanoparticle which is organic, alternatively inorganic, said inorganic nanoparticle being selected from the group comprising: mesoporous silica, organic polymers, protein-coated microcrystals, and MNPs.

There is also provided that the MNPs are coated-iron MNPs which are coated with silicon dioxide (SiO2), alternatively glutamic acid, further alternatively and most preferably Polyethylene Glycol (PEG).

It will be appreciated by a person skilled in the art that MNPs have magnetic properties and hence are recoverable, in use, from a reaction product using a magnetically active material.

In accordance with a third aspect of the invention there is provided a method of converting a waste oil to biodiesel, the method comprising utilizing the lipase having protein SEQ ID1 in a free alternatively and most preferably immobilised embodiment to catalyse the conversion of said waste oil to biodiesel in the presence of a short-chain alcohol in accordance with the following general reaction:

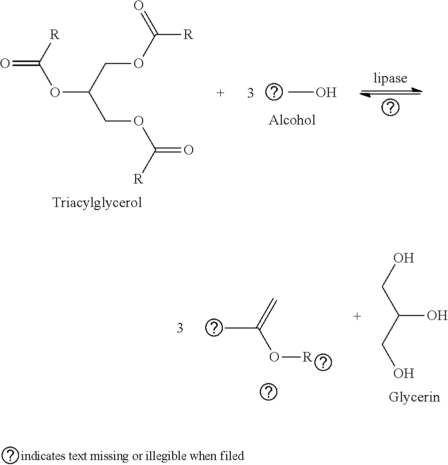

wherein R and R1 represent an alkyl chain of different lengths and/or saturation degrees.

There is also provided that the triacylglycerol is selected from the group comprising: saturated, monounsaturated, and polyunsaturated triacylglycerols and that the short-chain alcohol is selected from the group comprising: methanol, ethanol, butanol, propanol and amylic alcohol, most preferably methanol.

There is also provided that the waste oil is a plant-based alternatively, an animal-based waste oil.

There is also provided that the conversion of the waste oil feedstock to biodiesel takes place in a batch process, said process being conducted in a stirred-tank bioreactor, membrane bioreactor, fluidized bed reactor or packed-bed bioreactor. In accordance with a fourth aspect of the invention there is provided a lipase having protein SEQ ID1 encoded for by gene SEQ ID2 for use in a method of converting a waste oil to biodiesel, said method comprising contacting a waste oil feedstock with said lipase and allowing the same to catalyse the conversion of said waste oil to biodiesel in accordance with the method envisaged by the third aspect of the invention.

In accordance with a fifth aspect of the invention there is provided a nanoparticle to which a lipase having protein SEQ ID1 is adsorbed.

There is also provided that the lipase adsorbs to the nanoparticle via covalent conjugation.

There is further provided that the nanoparticle is a MNP, more specifically and coated iron-MNP, the same being coated with silicon dioxide (SiO2), alternatively glutamic acid, further alternatively and most preferably Polyethylene Glycol (PEG) and that, in use, the MNPs are recoverable from a reaction mixture by contacting the same with a magnetically active material.

In accordance with a sixth aspect of the invention there is provided a biodiesel product produced using the lipase or immobilised lipase of the first or second aspects of the invention and the methodology of the third aspect of the invention.

### BRIEF DESCRIPTION OF THE DRAWINGS

FIG. 1 shows the agarose gel electrophoresis of the linearized vector (pET30a+), PCR amplicon of the fragment/insert (lip-vut1) and the linearized pET30a+:lip-vut1;

FIG. 2 shows agarose gel electrophoresis of the linearized vector (pET30a+), PCR amplicon of the fragment/insert (lip-vut3) and the linearized pET30a+:lip-vut3;

FIG. 3 shows agarose gel electrophoresis of the linearized vector (pET30a+), PCR amplicon of the fragment/insert (lip-vut4) and the linearized pET30a+:lip-vut4;

FIG. 4 shows Agarose gel electrophoresis of the linearized vector (pET30a+), PCR amplicon of the fragment/insert (lip-vut5) and the linearized pET30a+:lip-vut5;

FIG. 5 shows agarose gel electrophoresis of the linearized vector (pET30a+), PCR amplicon of the fragment/insert (lip-vut7) and the linearized pET30a+:lip-vut7;

FIG. 6 shows agarose gel electrophoresis of the linearized vector (pET30a+), PCR amplicon of the fragment/insert (lip-vut9) and the linearized pET30a+:lip-vut9;

FIG. 7 shows SDS-PAGE of the induced protein wherein, M=Protein ladder, L 1 & 2 the induced Lip-vut1≈20 KD;

FIG. 8 shows SDS-PAGE of the induced protein wherein, M=Protein ladder, L 1 & 2 the induced Lip-vut3≈28 KD;

FIG. 9 shows SDS-PAGE of the induced protein wherein, M=Protein ladder, L 1 & 2 the induced Lip-vut4≈35 KD;

FIG. 10 shows SDS-PAGE of the induced protein wherein, M=Protein ladder, L 1 & 2 the induced Lip-vut5≈31 KD;

FIG. 11 shows SDS-PAGE of the induced protein wherein, M=Protein ladder, L 1 before induction, L2 & 3 the induced Lip-vut7≈28 KD;

FIG. 12 shows SDS-PAGE of the induced protein wherein, M=Protein ladder, L 1 before induction & L2 the induced Lip-vut9≈27 KD;

FIG. 13 shows SDS-PAGE of the induced protein, wherein L 1 Purified protein after dialysis Lip-vut1≈20 KD, M=Protein ladder;

FIG. 14 shows SDS-PAGE of the induced protein wherein M=Protein ladder, L 1 Purified protein after dialysis Lip-vut3≈28 KD;

FIG. 15 shows SDS-PAGE of the induced protein, wherein M=Protein ladder, L 1 Purified protein after dialysis Lip-vut4≈35 KD;

FIG. 16 shows SDS-PAGE of the induced protein wherein L 1 Purified protein after dialysis Lip-vut5≈31 KD, M=Protein ladder;

FIG. 17 shows SDS-PAGE of the induced protein wherein, M=Protein ladder, L 1 before induction, L2 & 3 after induction, L 4-6 first stage purification of Lip-vut7≈28 KD;

FIG. 18 shows SDS-PAGE of the induced protein wherein, L 1 Purified protein after dialysis Lip-vut9≈27 KD, M=Protein ladder;

FIG. 19 is a graph showing the effect of pH on the activity of recombinant lipases;

FIG. 20 is a graph showing the effect of temperature on the activity of recombinant lipases at their respective pH optima;

FIG. 21 is a graph showing the substrate affinity of recombinant lipases;

FIG. 22 is a graph showing the effect of alcohol on the activity of recombinant lipases;

FIG. 23 is a photograph of samples of biodiesel produced using 1: Lip-vut1, 2: Lip-vut3, 3: Lip-vut4, 4: Lip-vut5 and 5: Lip-vut9. negative control (-ve): Melted animal fat in methanol without lipase enzyme;

FIG. 24 is a computer-generated representation of the 3-D structure of Lip-vut1 lipase with two asymmetrical units. The crystal diffracted at a resolution of 1.63 A°;

FIG. 25 is a computer-generated representation of the 3-D structure of Lip-vut4 lipases. The crystal diffracted at a resolution of 2.15 A°;

FIG. 26 is a photograph of a first batch of a mini-scale synthesized magnetic nanoparticles (MNP): (A) Visual layer of the MNP at the bottom of the tube; (B) the MNP being attracted to the permanent magnet, as shown the particles moved to the side of the magnet;

FIG. 27 is a photograph of a second batch a medium scale synthesized magnetic nanoparticles (MNP): (A) Visual layer of the MNP at the bottom of the tube; (B) the MNP being attracted to the permanent magnet, as shown the particles moved to the side of the magnet;

FIG. 28 is a photograph of the reaction mixture and products in the formation of the MNP the orange solution (A) before the magnetic particles were formed and (B) After the magnetic particles were formed at different pH;

FIG. 29 is a photograph of the reaction mixture showing MNP products when exposed to a magnetic bar for a reaction run at pH 10. (A) Before the magnetic nano particles settled at zero time, (B) After the magnetic nano particles settled in 9 min & 3 s;

FIG. 30 is a photograph showing various samples containing MNP products. A 10-min timer was used to determine the strength of magnetic nanoparticles when exposed to a magnetic bar for a reaction run at pH 9. (A) Before the magnetic nano particles settled at zero time, (B) After the magnetic nano particles settled in 9 min & 3 s;

FIG. 31 is a photograph showing various samples containing the MNP products. A 10-min timer was used to determine the strength of magnetic nanoparticles when exposed to a magnetic bar for a reaction run at pH 7. (A) Before the magnetic nano particles settled at zero time, (B) After the magnetic nano particles settled in 9 min & 10 s;

FIG. 32 is a photograph showing the various samples of the MNP.A 10-min timer was used to determine the strength of magnetic nanoparticles when exposed to a magnetic bar for a reaction run at pH 6. (A) Before the magnetic nano particles settled at zero time, (B) After the magnetic nano particles settled in 9 min & 12 s;

FIG. 33 are TEM images of (a) uncoated magnetic nanoparticles (MNP) (b) Silicone oxide SiO₂-coated MNP;

FIG. 34 shows the Energy-Dispersive X-ray Spectroscopy (EDS) data analysis for the naked Fe₃O₄ nanoparticles;

FIG. 35 is the Fourier transform infrared (FTIR) spectra of the naked magnetic nanoparticles;

FIG. 36 shows the Energy-dispersive X-ray spectroscopy (EDS) data analysis for the SiO₂-coated magnetic nanoparticles;

FIG. 37 is the Fourier transform infrared (FTIR) spectra of the SiO₂-coated magnetic nanoparticles;

FIG. 38 is the Fourier-transform infrared (FTIR) spectra of Glutamic acid-coated MNP;

FIG. 39 is the Fourier-transform infrared (FTIR) spectra of PEG-coated MNP;

FIG. 40 shows Scanning Electron micrographs for SEM at 20 nm of nanoparticles coated with SiO₂ (A) and Polyethylene Glycol (PEG) (B);

FIG. 41 shows Scanning Electron Micrographs for SEM at 20 nm of nanoparticles coated with Glutamic Acid (A) and Polyethylene Glycol (PEG) (B);

FIG. 42 shows the FTIR spectra of the -MNP-PEG-Lipase complex;

FIG. 43 shows the FTIR spectra of the -MNP-PEG-glutaraldehyde-Lipase complex;

FIG. 44 shows the FTIR spectrum of biotin-lipase complex;

FIG. 45 shows the FTIR spectrum of immobilized biotinylated-lipase on streptavidin-MNP complex;

FIG. 46 is a graph showing a comparison of the relative activity of the immobilized vs free enzyme at different pH (4-10);

FIG. 47 is a graph showing a comparison of the relative activity of the immobilized vs free enzyme at different temperatures (30-70° C.);

FIG. 48 is a photograph showing samples showing the conversion of melted animal fat using immobilized enzyme at different methanol concentrations (40-100%);

FIG. 49 is a photograph of the reaction mixture of biodiesel production using melted animal fat and vegetable oil catalysed by the immobilized lipase;

FIG. 50 shows the FTIR spectrum of the biodiesel produced using vegetable oil as substrate;

FIG. 51 shows FTIR spectrum of the biodiesel produced using melted animal fat as substrate;

FIG. 52 shows the FTIR spectrum of Lip-VUT4 lipase isoform immobilized onto PEG-coated-magnetic nanoparticles;

FIG. 53 shows photographs of vessels containing produced biodiesel, the same being produced using immobilised Lipase 1 (a), immobilised lipase 2 (b) and free lipase (c);

FIG. 54 shows the FTIR spectrum of the biodiesel produced using immobilized and free lipase isoforms;

FIG. 55 shows photographs of vessels containing the reaction products using recycled MNP-immobilized lipases Lip-VUT4 (a) & Lip-VUT6 (b) for three cycles of biodiesel production;

FIG. 56 shows the FTIR spectra of the produced biodiesel;

FIG. 57 shows the FTIR spectra of the recycled MNP-enzyme complex recovered from cycle 3;

FIG. 58 is a computer-generated representation of the 3-D structure of Lip-vut4 lipases. The crystal diffracted at a resolution of 2.23 A°.

FIG. 59 shows the FTIR Spectrum of Fe₃O₄ nanoparticles coated with PEG;

FIG. 60 shows the FTIR spectrum of Lip-VUT4 immobilised on PEG-coated iron oxide magnetic nanoparticles;

FIG. 61 shows photographs of vessels containing the biodiesel produced (B) using immobilised Lip-Vut 4, from a mixture of waste animal fat and methanol and (A) prior to adding the lipase;

FIG. 62 shows the FTIR spectrum of the synthesized biodiesel;

FIG. 63 is a graph showing the activity of recovered immobilised lipase after each cycle;

FIG. 64 shows chromatograms resulting from FTIR analysis of the sample;

FIG. 65 Gas chromatograms of fatty acid methyl esters of the produced biodiesel, including the internal fatty acid methyl ester standard mixture;

FIG. 66 shows FTIR spectra of the synthesized magnetic nanoparticles (in black) and the PEG-coated magnetic nanoparticles (in red);

FIG. 67 shows photographs of vessels containing the biodiesel produced using unused palm oil (FIG. 67a) and waste palm oil (FIG. 67b) wherein the upper layer comprises biodiesel and the lower layer comprises a combination of glycerol, lipase and unconverted oil; and

FIG. 68 shows the FTIR spectra of biodiesel produced from waste palm oil (FIG. 68a) and clean palm oil (FIG. 68b) using the immobilised enzyme as the catalyst.

### EXAMPLES

The invention will be exemplified with reference to the examples given hereunder. However, it will be appreciated by a person skilled in the art that where standard biochemical methods are used and the same may be replaced by alternative methodology that the scope of the examples will extend to such methodology.

### Example 1: Clonina of Lipase Genes

Six lipase genes denoted lip-vut1, lip-vut3, lip-vut4, lip-vut5, lip-vut7 and lip-vut9 were cloned into pET30a+ vector using standard cloning techniques applicable in the field of molecular biology. Successful cloning was confirmed via PCR (Polymerase Chain Reaction) to amplify the fragments/inserts of the genes of interest and subsequent electrophoresis of the linearized vector and PCR amplicon was performed.

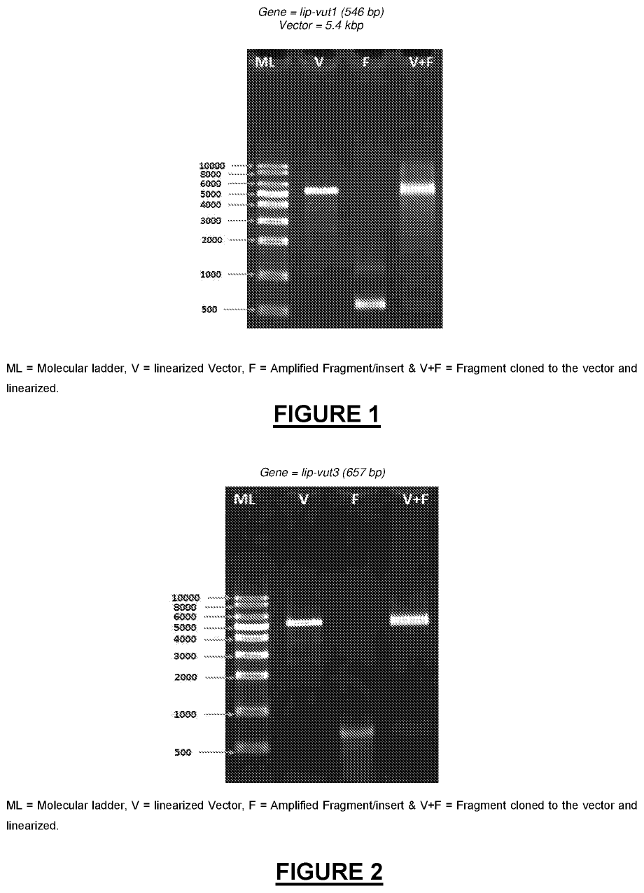

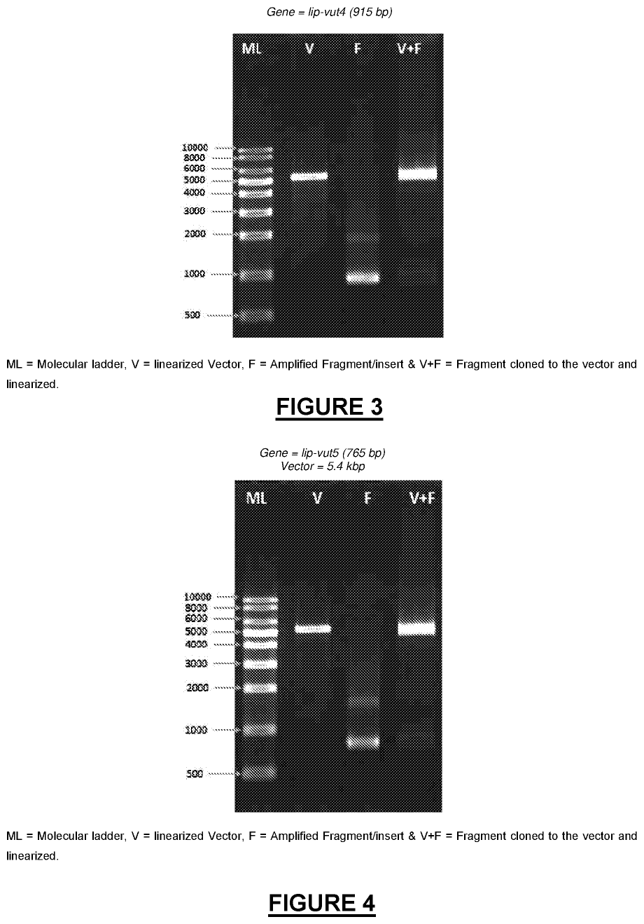

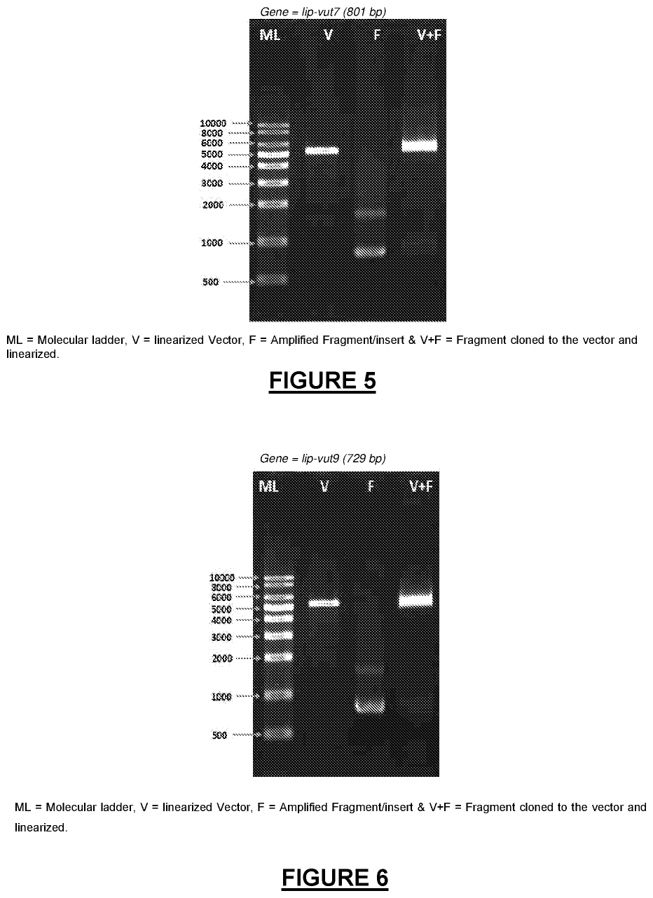

FIGS. 1 to 6 show the results of agarose gel electrophoresis of the linearized vector (pET30a+), PCR amplicons of the fragments/inserts, which were genes of interest, and the linearized pET30a+: lip-vut1 (FIG. 1), pET30a+:Iip-vut2 (FIG. 2), pET30a+:Iip-vut4 (FIG. 3), pET30a+: lip-vut5 (FIG. 4), pET30a+: lip-vut7 (FIG. 5), and pET30a+:lip-vut9 (FIG. 6).

### Example 2.1: Protein Expression

The proteins of all six recombinant genes (lip-vut1, lip-vut3, lip-vut4, lip-vut5, lip-vut7 and lip-vut9) cloned into pET30a+ were expressed and purified. E. coli BL21 was transformed with recombinant plasmids. A single colony was inoculated into LB medium containing kanamycin; culture was incubated at 37° C. shaken at 200 rpm and then induced with IPTG. Then the induced proteins were partially purified using Akta Protein Purification system and loaded onto (SDS-PAGE sodium dodecyl sulfate-polyacrylamide gel electrophoresis).

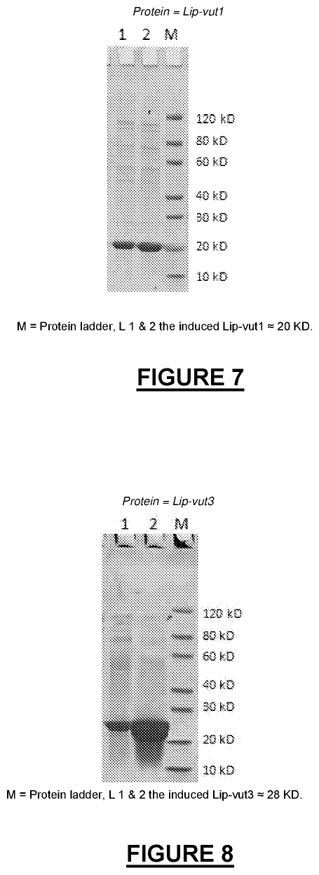

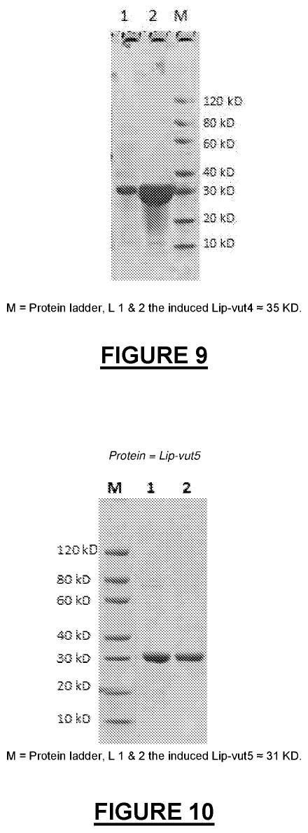

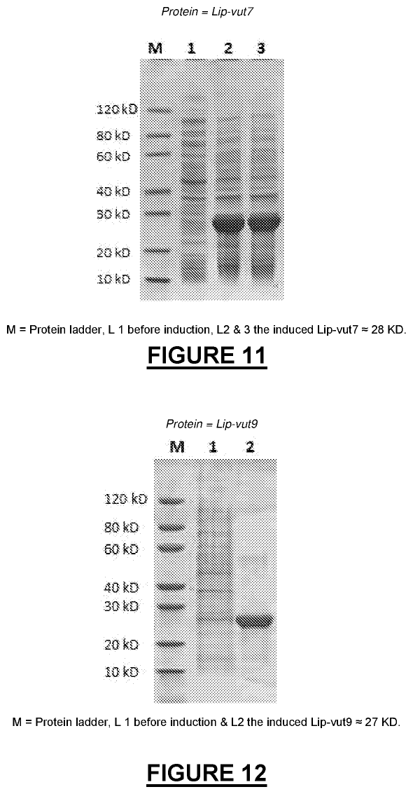

The results of the SDS-PAGE are shown in FIGS. 7 to 12 wherein each of the induced proteins is shown using the nomenclature related to the cloned gene:

### Example 2.2: Protein Purification

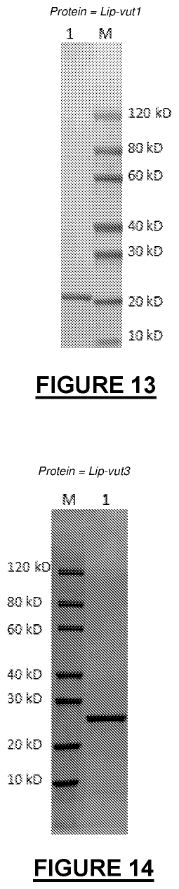

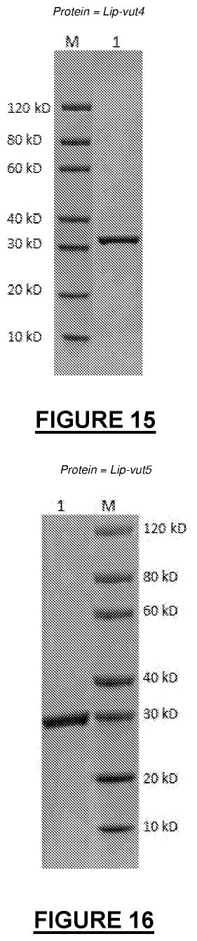

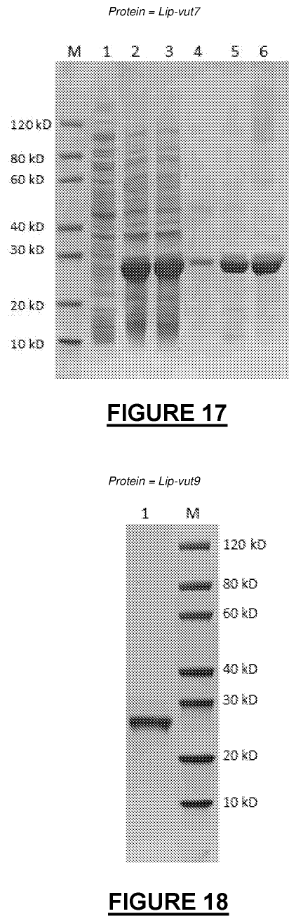

As above, E.coliBL21 was transformed with recombinant plasmids. A single colony was inoculated into LB medium containing kanamycin; culture was incubated in 37° C. at 200 rpm and then induced with IPTG. SDS-PAGE electrophoresis was then conducted on samples A-D & F referred to above after dialysis. The results of the same are shown in FIGS. 13-18.

### Example 3: Stability Studies

The effects of pH, temperature and methanol on the candidate lipases was assessed as was the substrate affinity for the same.

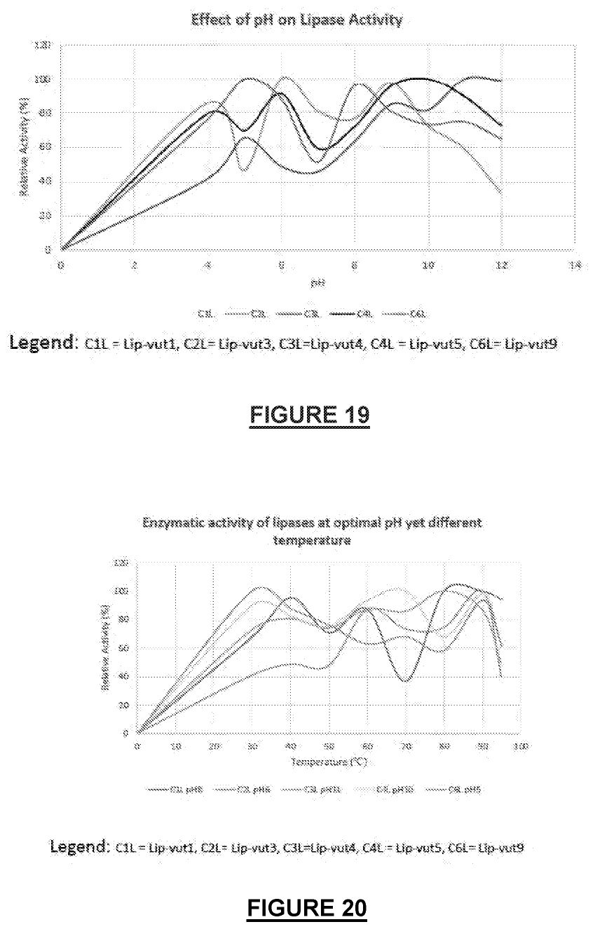

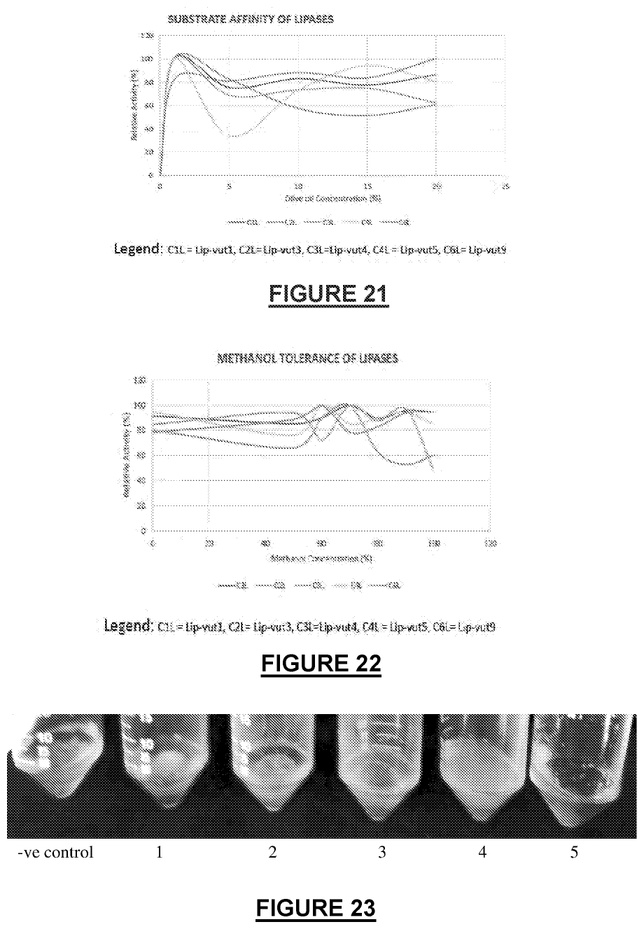

Results: The results of these studies are shown in FIGS. 19 to 22 and Table 1 which summarises the optimum conditions for the lipases.

TABLE 1
Summary of the optimal conditions.

| | Optimum temp | Optimum |
|---|---|---|
| Lipases** | (° C.) | pH |
| Lip-vut1 | 80 | 8 |
| Lip-vut3 | 90 | 6 |
| Lip-vut4 | 80 | 11 |
| Lip-vut5 | 70 | 10 |
| Lip-vut9 | 30 | 5 |

The candidate recombinant lipases largely maintained 80% of their activity at different substrate and alcohol concentration. Based on the data from physico-chemical characterization Lip-vut1, Lip-vut3, Lip-vut4, and Lip-vut5 were found to be good candidates for further characterization and downstream application.

### Example 4: Preliminary Tests of the Candidate Lipases for Biodiesel Production

Preliminary tests of the candidate lipases for biodiesel production were conducted and the results of the same are shown in FIG. 23.

FIG. 23 shows that the animal fat has been broken down as there is a formation of two clear layers: a viscous one at the bottom, presumptive glycerol, which is the by-product of catalytic production of biodiesel and the top layer, presumptive biodiesel with some residues of water and remaining methanol.

### Example 5: Physiochemical Studies

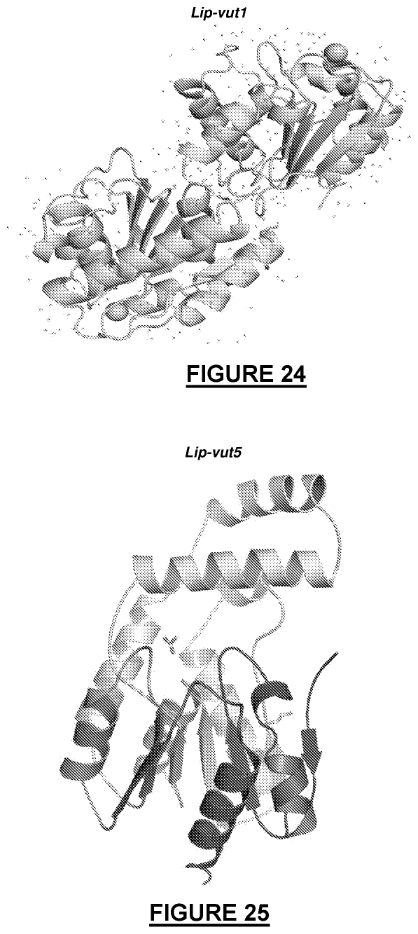

E. coli BL21(DE) was transformed with plasmids carrying lipase genes. Then expressed and purified using AKTA Express protein purification system. Then, the proteins were concentrated, and crystallization experiment was set up under different screening conditions, then screened for formation of crystals. The formed crystals were picked & cryo-frozen until the same could be exposed to beam line at a facility for Advanced Photon Source. The results of these studies are shown hereunder in FIGS. 24 and 25 and the nomenclature for the samples remains the same.

### Example 6: Immobilisation of the Lipases on a Magnetic Surface

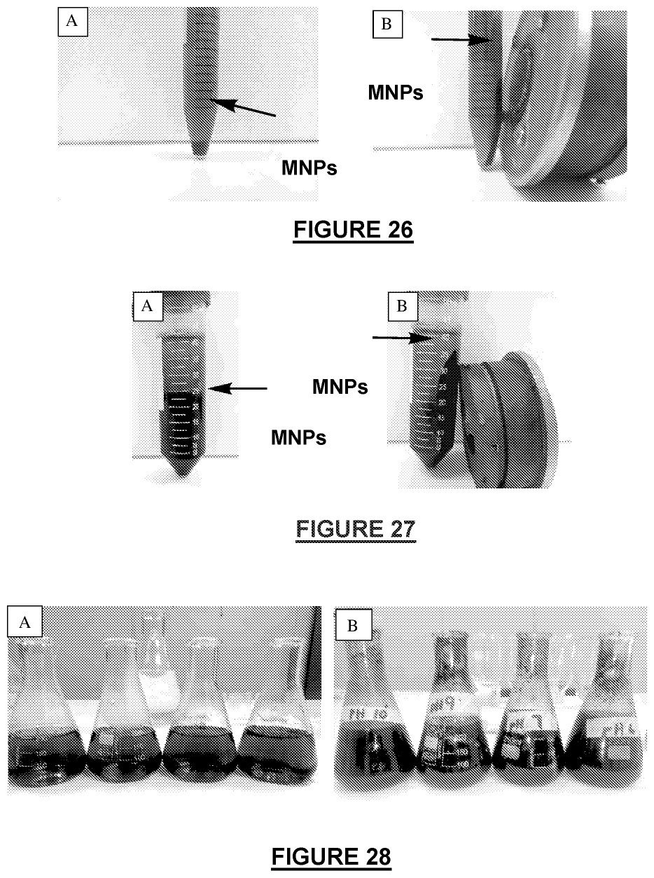

The first batch of the magnetic nanoparticles were synthesized as summarized (FIG. 26). Then production of the same was scaled up (FIG. 27) and synthesized at a relatively medium scale. The next step following the medium-scale synthesis will be to synthesize the magnetic nanoparticles (MNP) at a relatively larger scale. Then, to probe the stability of the MNP under different physico-chemical condition of the reactor.

### Example 7: Studies of the Stability of the Magnetic Nanoparticles at Different pH

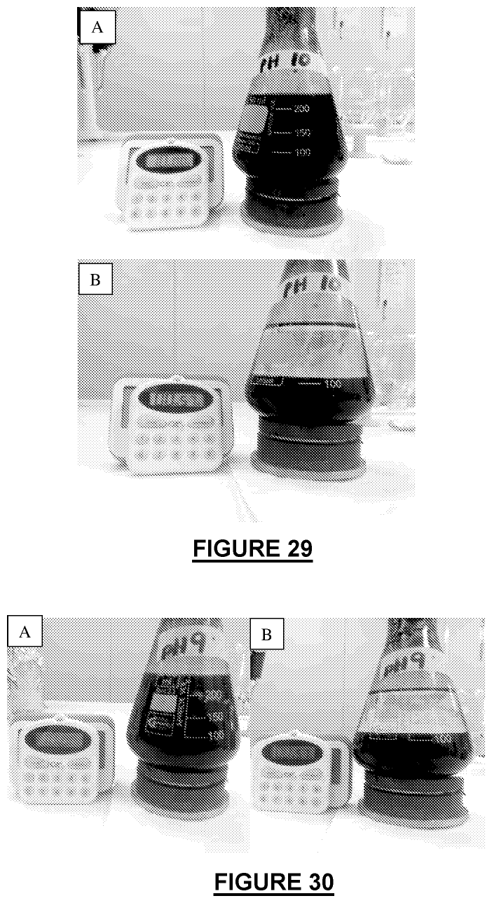

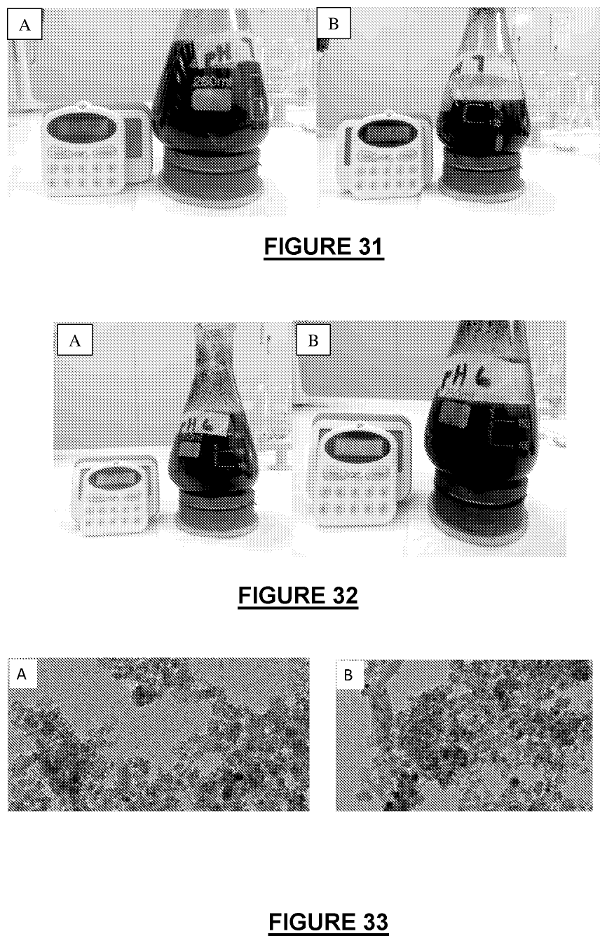

The stability of the MNP under different pH conditions of the reaction were performed. The results obtained are shown in the FIGS. 28 to 32.

The magnetic nano particles (MNP) synthesized at different pH showed relatively similar magnetic properties at different pH. However, the MNP synthesized at relatively alkaline pH showed better magnetic property. Since, biodiesel is formed at alkaline pH, the relatively high magnetic property displayed by the MNP was shown to be promising for the intended downstream application.

### Example 8: Stabilisation of Magnetic Nanoparticles

Give that magnetic nano particles are very reactive in nature and get agglomerated, they will lose their magnetic property unless stabilized using coating agents. As such, different coating agents were considered and characterized as agents to stabilize the MNP whilst maintaining their magnetic properties.

### Synthesis and Coating of MNP with PEG

The magnetic nanoparticles were synthesized and coated with polyethylene glycol (PEG). For the coating of nanoparticles, ratio of MNPs to PEG was 1:1. When adding PEG, the solution was continuously stirred, the reaction was run at 30° C. for 2 hours. The MNPs were washed with distilled water and ethanol to remove impurities. The nanoparticles were later dried in an oven at 50° C. for 1 hour.

It was found that the resulting colour of the solution after adding PEG remained the same.

Coating of MNPs with Silicon Oxide (SiO₂)

The magnetic nanoparticles were synthesized and coated with SiO₂ as stated below. The coated MNPs were examined using Transmission Electron Microscopy (TEM). The results of the TEM are shown in FIG. 33.

Results: the magnetic nanoparticles were successfully coated with silicone oxide (SiO₂) as confirmed with TEM analysis (FIG. 33). The coated nanoparticles showing relatively better dispersal (FIG. 33b) than the uncoated nanoparticles (FIG. 33a).

### Example 9: Characterisation of the Synthesized Magnetic Nanoparticles

The synthesized naked and SiO₂-coated magnetic nanoparticles were characterized using Energy-dispersive X-ray spectroscopy (EDS) and Fourier transform infrared (FTIR) spectra.

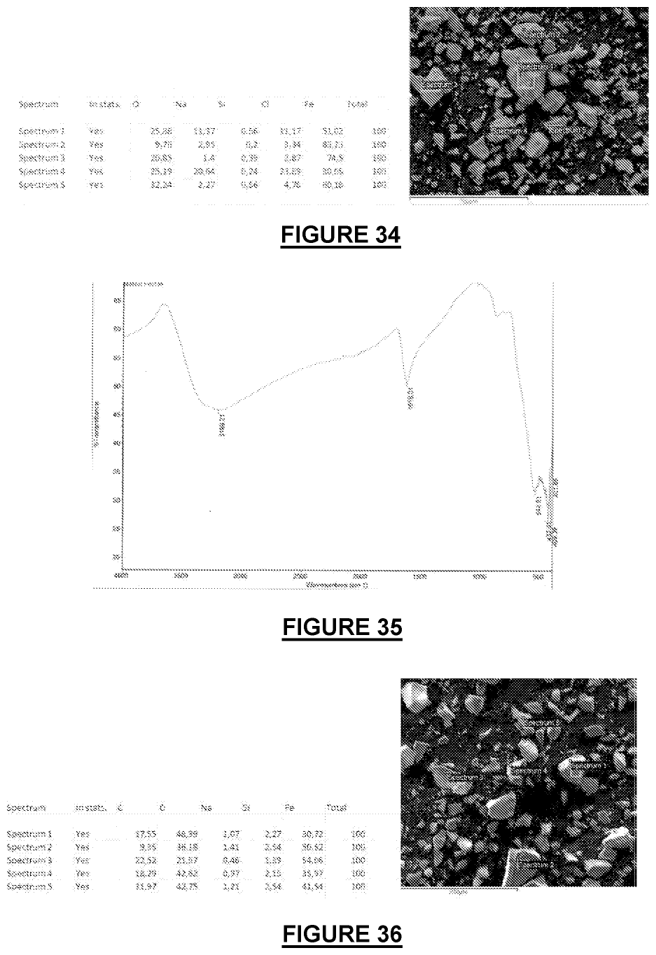

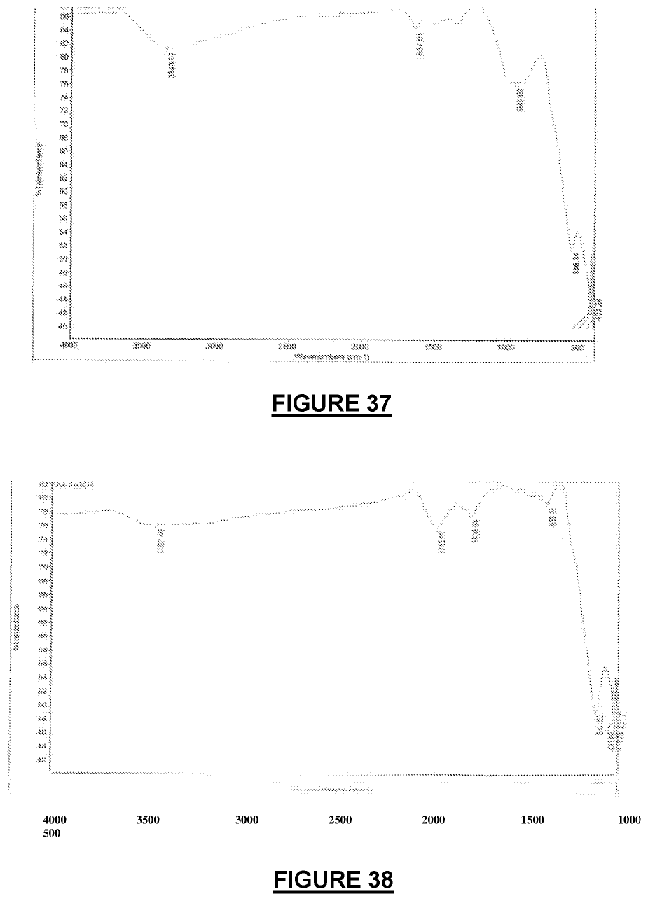

The results of the characterisation of naked magnetic nanoparticles using Energy-Dispersive X-ray Spectroscopy (EDS) and Fourier Transform Infrared (FTIR) are shown in FIGS. 34 to 39.

The data obtained shows the presence of Fe and O in abundance, thereby confirming the formation of iron oxide nanoparticles.

The data obtained illustrated the presence of Fe—O bond, showing in 544.61 cm⁻¹ wavelength. This peak confirms the formation of iron oxide nanoparticles (FIG. 35).

Characterisation of SiO₂-coated magnetic nanoparticles using Energy-Dispersive X-ray Spectroscopy (EDS) and Fourier Transform Infrared (FTIR).

A given sample should be surface-charged before it is exposed to Scanning Electron Microscopy (SEM) so that the sample emits photons when bombarded by the beam of the SEM. Accordingly, some samples are surface-charged by default because of physicochemical characteristics of the particular sample as in FIG. 34 and some are not as in FIG. 36, hence surface-charged by coating with charcoal as indicated by the percentage of carbon indicated in the table of FIG. 36.

According to the data the silane coating is present, even though it is in small quantities. The amount of Si found within the different spectrum has increased in comparison to the data from naked Fe₃O₄ (FIG. 34).

The wave number 556.45 cm⁻¹ of the peaks represent the Fe—O bond found in the magnetite nanoparticle (FIG. 3). The peak at 946.02 cm⁻¹ wavelength indicates the presence of Si—O—Si bond in the crystals furthermore this clearly shows that the nanoparticles have been successfully coated with silane. The amine (NH₂) which was brought by coating with APTES is indicated at 3343 cm⁻¹ and 1637 cm⁻¹, according to FTIR spectra database primary amine-stretch (NH₂) peaks show between 3400-3250 cm⁻¹ wavelength, whereas primary amine-bond have wavenumber between 1650-1580 cm⁻¹. In this case, it verified that the amino group was free (not bound to anything), and implied that, in future the enzyme could be successfully immobilised through such bonding via free amino group to form a diazote bond (support-N═N-enzyme).

Therefore, the coating stabilized the MNP and did not interfere with the free amino group needed for immobilisation of the enzyme.

Characterisation of magnetic nanoparticles (MNP) coated with Glutamic acid and polyethylene glycol (PEG) using Fourier Transform Infrared (FTIR) and scanning electron microscopy (SEM) The Fourier-transform infrared (FTIR) spectra of the magnetic nanoparticle coated with glutamic acid is shown in FIG. 38.

The FTIR analysis of the Glutamic acid-coated MNP was done in order to correctly identify the presence or absence of the expected bond in the Fe₃O₄-MNP as mentioned earlier besides other bonds depending on the type of coating materials used.

According to the analysis the Glutamic acid-coated MNP maintained Fe—O bond as evidenced by the peak at 540 cm⁻¹ wave number (FIG. 38). The finding was consistent with the XRD result.

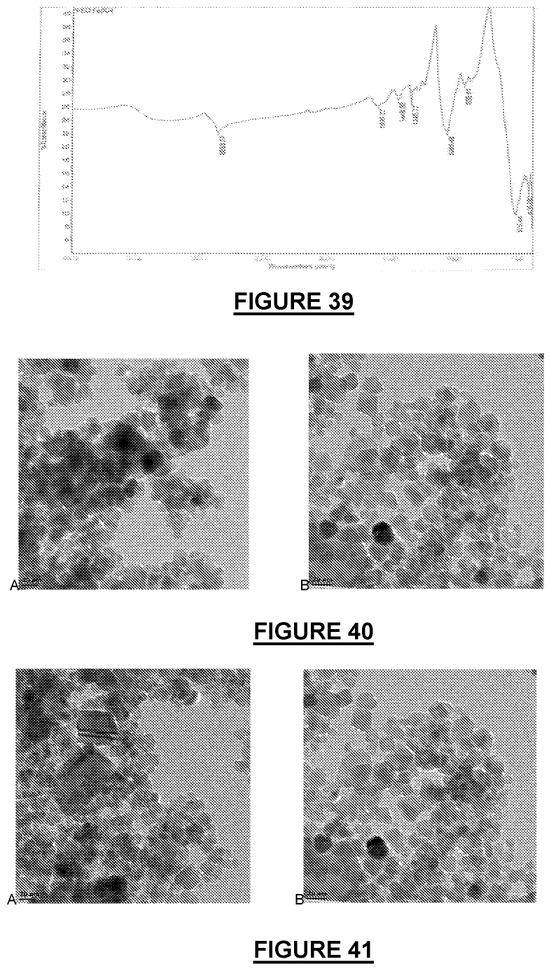

The FTIR analysis showed that the PEG-coated MNP maintained Fe—O bond as evidenced by the peak at 529 cm⁻¹ wave number (FIG. 39). The finding was also consistent with the XRD result.

### Example 10: Scanning Electron Microscopy (SEM) Analysis of the Coated MNP

The Scanning Electron Microscopy (SEM) Analysis of the MNP coated with either SiO₂, Polyethylene Glycol (PEG) or glutamic acid was done in order to investigate if the coating had reduced the agglomeration of the MNP, which is common in non-coated or naked MNP. A good coating would be expected to give a relatively dispersed MNP. The dispersion of the MNP is critical as it increases the surface area of the MNP and hence increases the number and exposure of immobilised enzyme per MNP, thereby influencing the catalytic efficiency of the immobilised enzymes.

The SEM analysis showed that the MNP coated with polyethylene glycol (PEG) had been relatively dispersed and was homogenous in size and shape as compared to the MNPs coated with SiO₂ or glutamic acid (FIG. 40 & 41).

X-ray diffraction (XRD) results confirmed that for all coating techniques employed that the MNPs did not lose their magnetite properties. However, the coating affected polydisperity, shape and size of the MNPs at different degrees. Hence, the coating of the magnetic nanoparticles with Polyethylene Glycol (PEG) showed a relatively well-dispersed coated-MNPs and a relatively homogenous and comparable size as compared to the glutamic acid and SiO₂ coated-MNP. As such, PEG was identified as the most favourable coating for the coating of the MNPs before the immobilisation of the enzyme.

### Example 11: Immobilization of Lipase on Polyethylene Glycol-Coated Magnetic Nano Particles (MNP) Via Covalent Coniugation

Lipase enzyme was covalently conjugated on Magnetic Nanoparticle (MNP) coated with Polyethylene Glycol (PEG).

### Example 11.1. Immobilization of Lipase on Polyethylene Glycol-Coated Magnetic Nano Particles (MNP) Via Covalent Coniugation in the Absence of Glutaraldehyde

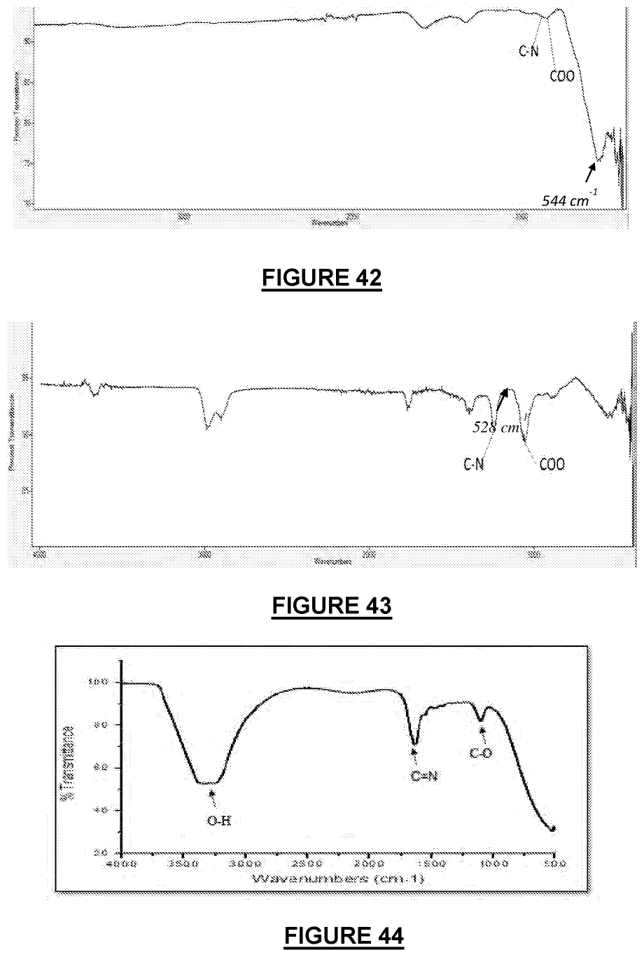

The FTIR spectrum (FIG. 42) shows different peaks of functional groups found within the reaction mixture. The absorption bands around 1100 cm⁻¹ originate from stretching vibrations of C—N, which is formed by the bond between the lipase and the PEG-coated MNPs. The peak at 544 cm⁻¹ represents the presence of strong Fe—O bond, which results from the MNPs, showing that the MNP is intact.

The nanoparticle did not lose its magnetite property as the result of enzyme immobilization. A sharp —COO stretch around 1066 cm⁻¹ was observed in the spectra and the peak was produced from the vibration of the carboxyl group formed as the MNPs and PEG was bonded.

### Example 11.2. Immobilization of Lipase on Polyethylene Glycol-Coated Magnetic Nano Particles (MNP) Via Covalent Coniugation in the Presence of Glutaraldehyde

The reaction was conducted to determine how the PEG and the enzyme would bond in the presence of another agent, glutaraldehyde. The FTIR spectra show different peaks which was expected. The C═N functional group vibration is picked up at around 1241 cm⁻¹ as a result of the bonding of the enzyme to PEG-coated MNPs. The Fe—O bonds peak is also visible around 528 cm⁻¹ wavelengths, which results from the MNPs, showing that the MNP is intact and did not lose its magnetite property as the result of enzyme immobilization in the presence of glutaraldehyde (FIG. 43). The peak around 1065 cm⁻¹ represents the carboxyl group between the MNPs and the PEG. A peak was expected to be visible around 1690-1640 cm⁻¹, which would have been a vibration from the stretching C═N bond that is found within the crosslinked enzymes, because glutaraldehyde was expected to cross link the enzymes. The absence of the C═N indicates that glutaraldehyde neither reacted with the enzyme nor did it interfere with the immobilization of the enzyme on the PEG-coated MNP.

Therefore, lipase was successfully immobilized on PEG-coated MNP both in the absence and presence of glutaraldehyde and the glutaraldehyde neither cross-linked the enzyme to one another nor did it interfere with the immobilization of the enzyme on the PEG-coated MNP.

### Example 11.3. Immobilization of Lipase on Biotinylated Streptavidin Magnetic Nanoparticles (MNP) Via Biologically Mediated Specific Interaction

The bond between Biotin and Streptavidin is one of the strongest non-covalent bonds that exists. The biotin-streptavidin coupling is normally used to couple two different molecules that would not traditionally bind to each other. Accordingly, MNP was attached to the lipase through the biotin-streptavidin complex.

Lipase was coupled with biotin prior to attaching the biotin onto the streptavidin coated MNPs. Accordingly, the FTIR spectrum showed the presence of C═N group that originates from the bond between lipase and the biotin. The C—O functional group that is represented at peak 1108 cm⁻¹ was from the carboxyl group found in biotin molecule. The broad peak between 3250 and 3500 cm⁻¹ resulted from the vibrations of O—H which was present in the carboxyl functional group of biotin (FIG. 44).

Immobilisation of Biotin-Bound Lipase on the MNP Coupled with Streptavidin

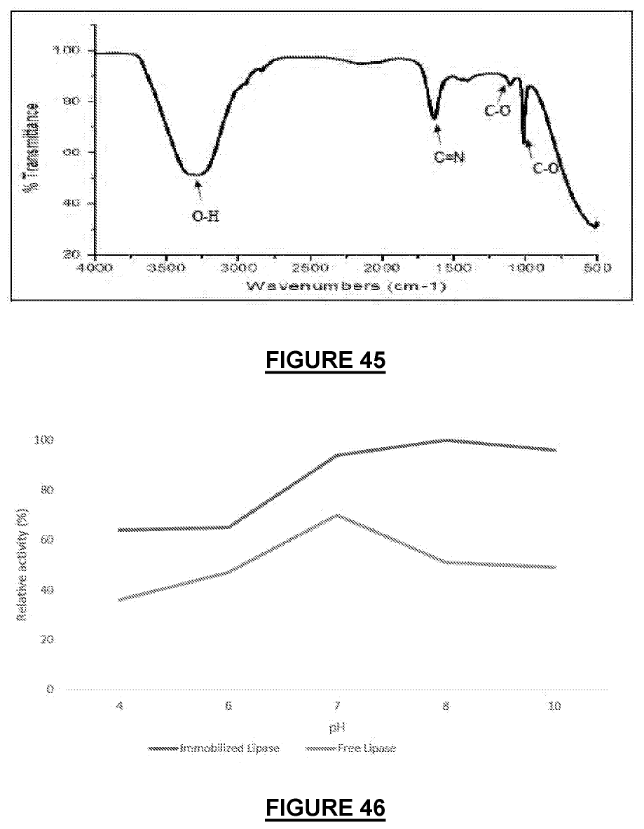

The results for this experimentation are shown in FIG. 45. The presence of a strong sharp peak that appeared at exactly 1015 cm⁻¹ representing the C—O functional group, confirms the formation strong bond between the biotin and streptavidin. Hence, the biotinylated-lipase was successfully anchored on streptavidin-MNP complex.

### Example 12: Physicochemical Characterization of the Immobilized Lipase

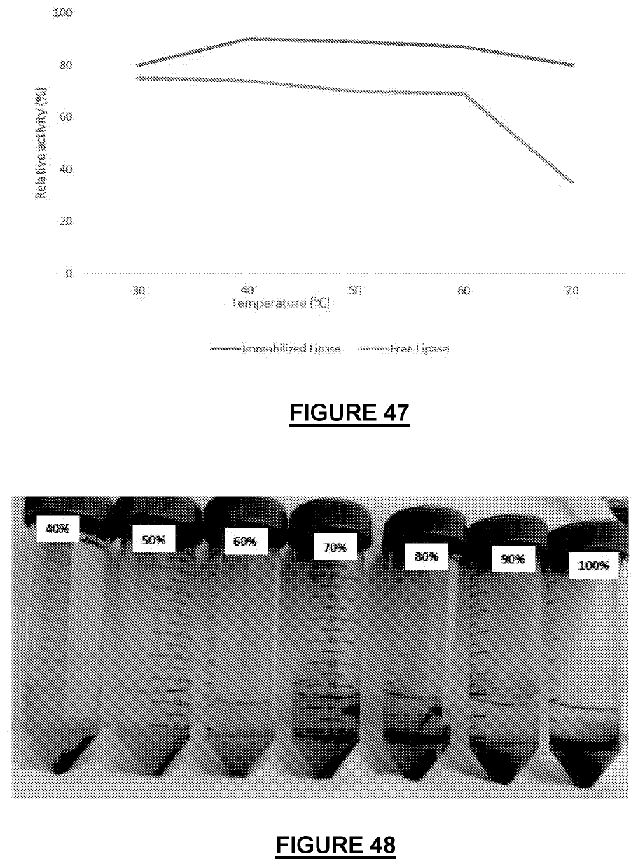

The biotinylated-lipase anchored on streptavidin-MNP complex showed a relatively strong bond, hence stability, as compared to other immobilization techniques. Hence, the biotinylated-lipase anchored on streptavidin-MNP complex was further characterized for its activity at different pH (4-10), temperature (30-70° C.) and substrate preference (animal fat vs pure vegetable oil). The results of this characterisation are shown in FIGS. 46, 47 and 48.

Accordingly, the immobilized enzyme maintained its activity above 60% at low pH while the activity peaked at pH 8 and above 94% of the activity was maintained between pH 7-10. The fact that the maximum activity was seen at pH 10 shows that the enzyme can tolerate alkaline conditions.

Alkaline tolerance is very critical as it implies that the immobilized enzyme could be used in the same reactor that was setup for production of biodiesel using alkaline catalyst (NaOH or KOH). On the other hand, the free lipase peaked at pH 7 then started to decline as the pH increased. Therefore, the result showed that immobilization of the enzyme significantly increased the activity and stability of the enzyme at different pH as compared to the free lipase.

Accordingly, the relative activity of the immobilized enzyme peaked at 40° C. and 80% of the activity was maintained even if the temperature was increased to 70° C. This shows that immobilization rendered the enzyme improved activity and stability at elevated temperature as opposed to the free enzyme. The improved stability at elevated temperature has positive implication on the shelf life of the immobilized enzyme.

The immobilized lipase showed stability at different concentration of methanol (40-100%), successfully converting the melted animal fat to biodiesel at all concentrations of methanol tested (FIG. 48).

### Example 13: The Substrate Preference of the Immobilised Enzyme for the Production of Biodiesel

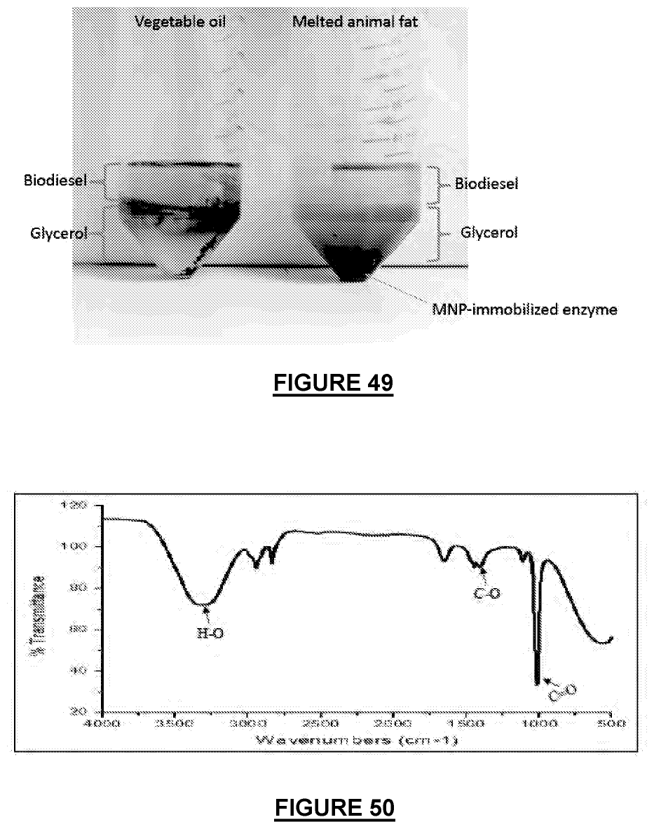

In order to understand the substrate preference of the immobilised enzyme melted animal fat and vegetable oil was used as raw materials. Accordingly, the immobilized enzyme successfully converted both raw materials to biodiesel. The glycerol by-product that was produced when vegetable oil was converted being clearer than the one from melted animal fat (FIG. 49).

At the end of the reaction, three distinct layers were formed after centrifugation representing biodiesel, glycerol, and MNP-immobilized enzymes, respectively (FIG. 49). The biodiesel produced using both raw materials was further characterized using FTIR.

### Example 14: Characterisation of the Synthesized Biodiesel Using FTIR

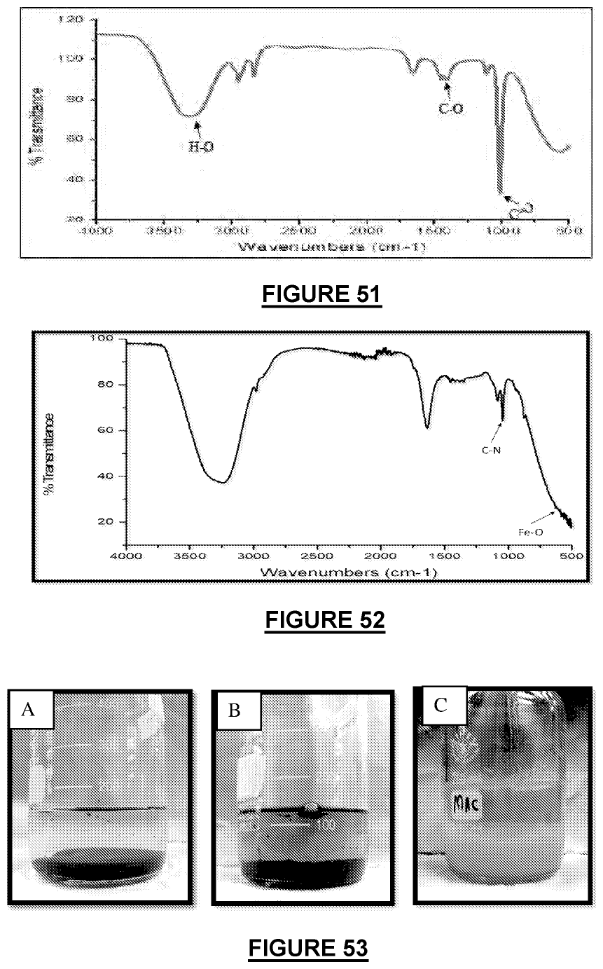

The synthesized biodiesel (synthesized using vegetable oil as a substrate) was characterized using FTIR and the results of the same are shown in FIG. 50 (synthesized using vegetable oil as a substrate) and FIG. 51 (synthesized using melted animal fat as a substrate).

FIG. 50: The spectrum shows the functional group of the biodiesel produced using vegetable as the substrate. This depicts the presence of an ester with C═O and C—O by a peak around 1000 cm⁻¹. The peak around 1400 cm⁻¹ represents the vibrations of methyl ester (CH₃—O—C(═O)—R₂) found within the biodiesel. This confirms that the product synthesized was biodiesel.

FIG. 51: The biodiesel produced using melted animal fat as a substrate resembles that of biodiesel from vegetable oil (FIG. 50). This illustrates that the lipase can use both refined and unrefined oils as a substrate.

The lipase was successfully immobilized via biologically mediated specific interactions. The immobilization of the enzyme significantly improved the pH tolerance, thermostability and alcohol tolerance of the enzyme. The enzyme was also not feedstock specific in that it successfully converted vegetable oil and melted animal fat to biodiesel.

### Example 15: Gas Chromatography—Flame Ionization Detector (GC-FID) Studies of the Fatty Acid Methyl Esters (FAMEs) in the Biodiesel Samples

This example relates to the analysis of two different samples of biodiesel produced in accordance with the method of the invention.

### Sample Preparation:

2 ml (2:1 chloroform: methanol) was added to ca. 250 mg sample. The sample was vortexed and sonicated at room temperature for 30 minutes. The sample was centrifuged at 3000 rpm for 1 minute. 200 μl of the bottom layer (chloroform) was dried completely with a gentle stream of nitrogen and reconstituted and vortexed with 170 μl of methyl tert-butyl ether (MTBE) and 30 μl of trimethylsulfonium hydroxide (TMSH). 1 μl of the derivatized samples was injected in a 5:1 split ratio onto the GC-FID.

### Chromatographic Separation:

Separation was performed on a gas chromatograph (6890N, Agilent Technologies) coupled to flame ionization detector (FID). Separation of the FAMEs was performed on a polar RT-2560 (100 m, 0.25 mm ID, 0.20 μm film thickness) (Restek, USA) capillary column. Hydrogen was used as the carrier gas at a flow rate of 1.2 ml/min. The injector temperature was maintained at 240° C. 1 μl of the sample was injected in a 5:1 split ratio. The oven temperature was programmed as follows: 100° C. for 4 minutes, ramped to 240° C. at a rate of 3° C./min for 10 minutes. Table 2 and 3 hereunder showing the samples registered and the results associated with the samples.

TABLE 2
is the sample registration form for the samples analysed
Sample information

| Sample | | |
|---|---|---|
| Number: | Sample Description | Lab Number (CAF Use) |
| 325 | Biodiesel | 2022-53 [symbol] |
| 326 | Biodiesel recycled | 2022-53 [symbol] |

[symbol]

indicates data missing or illegible when filed

TABLE 3
shows the results associated with the analysis of the samples

| | Compound (FAMEs) and concentration in mg/g | | | | | | | | | | | | | | | | | |
|---|---|---|---|---|---|---|---|---|---|---|---|---|---|---|---|---|---|---|
| | | | | | | | | | | | | | | | C20:1 | | | |
| Sample | | | | | | C16: | | C17: | | C18: | C18: | C18: | | C18: | and | | | |
| name | C8 | C12 | C14 | C15 | C16 | 1 | C17 | 1 | C18 | 1cis | 2trans | 2cis | C20 | 3n6 | C18:3n6 | C20:2 | C22 | C24 |
| Biodiesel | 0.028 | [symbol] | 0.063 | 0.018 | 2.94 | [symbol] | 0.074 | 0.011 | 1.30 | [symbol] | [symbol] | [symbol] | [symbol] | [symbol] | [symbol] | 0.011 | [symbol] | [symbol] |
| Biodiesel | 0.052 | [symbol] | [symbol] | 0.021 | 3.96 | 0.056 | 0.064 | 0.014 | 1.64 | [symbol] | [symbol] | [symbol] | 0.067 | 0.037 | [symbol] | 0.012 | 0.109 | 0.025 |
| recycled | | | | | | | | | | | | | | | | | | |

FAMEs carbon number and commons names:
C8-methyl octanpoate
C12-methyl laurate
C14-methyl myristste
C16-methyl pentaderanoate
C16-methyl palmitate
C15:1-methyl palmitoleate (cis-9)
C17-methyl heptadecanoate
C17:1-methyl heptadecenoate (cis-10)
C18-methyl stearate
C18:1cis-methyl oleate (cis-90
C18:2trans-methyl linoleaidate (trans-9,12)
C18:2cis-methyl [symbol] (cis-9.32)
C20-methyl arachidate
C18:3n6-methyl y-linolenate (cis 6,9,12)
C20:1-methyl eicosenoate (cis-3.1)
C18:3n3-methyl itnotenate (cis-9,12,15)
C20:2-methyl eicatsdiencate (cis-11,34)
C22 - methyl betenate
C24-methyl lignocesate [symbol]
indicates data missing or illegible when filed

The above tests results show that the immobilised lipase in accordance with the invention demonstrated a high capacity with respect to catalysing transesterification reactions using waste animal fat as a substrate obtaining FAME conversion of 58.74+/−0.69% in a four-hour reaction. The enzymes also showed over 60% activity after recycling.

### Example 16: Optimisation of the Method of Producing Biodiesel Using 100 ml of the Immobilised Enzyme

Having found that Lip-VUT4 lipase isoform showed the most promise for commercial application all further tests were conducted using the same

### Example 16.1: Preparation of Immobilised Lipase Isoforms

The Lip-VUT4 lipase isoform was immobilised on PEG-coated-MNP and FTIR was used to analyse the same. The results of this analysis are shown in FIG. 52.

The resulting FTIR spectrum in respect of the immobilized lipase showed the presence of C—N bond, which was formed between the coated MNP, and lipase and the Fe—O bond showed that the MNP was intact.

### Example 16.2. In Vitro Production of 100 ml Biodiesel

The transesterification reaction was conducted at 30° C. for 24 hours to achieve complete conversion. The reaction batch consisted of fat, methanol, and enzyme. The produced biodiesel (FIG. 53) with the dark MNP-immobilized enzyme sediment at the bottom.

With reference to FIG. 53: The top layer in each of the photographs consists of the biodiesel, whereas the bottom layer consists of glycerol and the immobilised lipase (immobilised lipase in Photographs 53(a) and 53(b)). As can be seen in FIG. 53 conversion of the melted animal fat to biodiesel was incomplete when the free enzyme was used FIG. 53(c). In FIG. 53(c), the clear layer located at the top of the vessel comprises biodiesel and glycerol while a layer of animal fat is visible at the bottom of the vessel. The presence of unhydrolyzed animal fat shows incomplete conversion of feedstock to biodiesel in the presence of the free enzyme.

### Example 16.3: Characterisation of the Synthesized Biodiesel Using FTIR

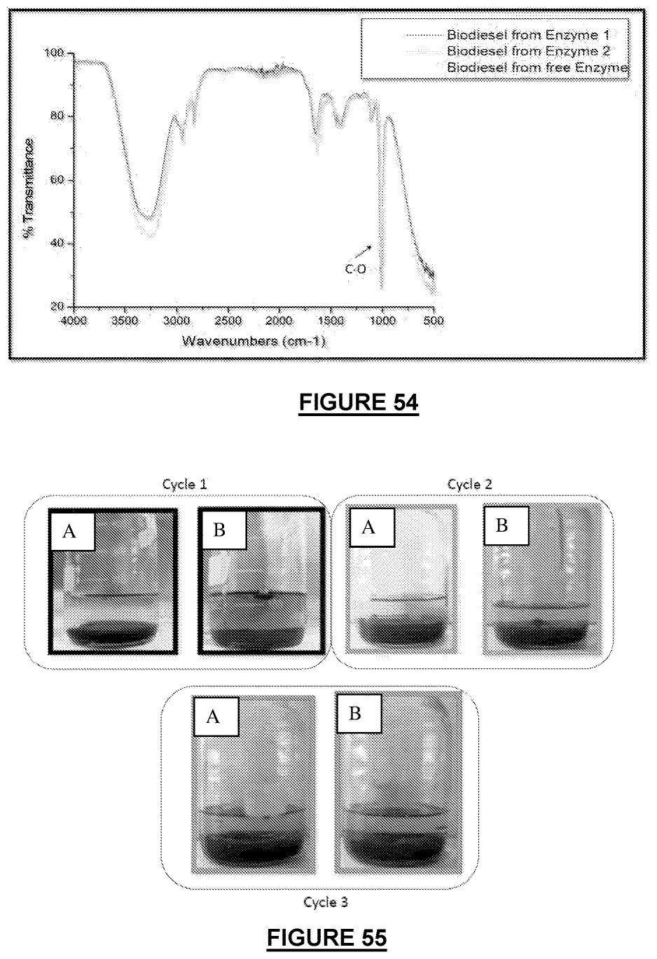

The synthesized biodiesel was characterised using FTIR. The results of the characterisation being shown in FIGS. 54, wherein the three spectra showed similarities in all aspects. The peak at 1010 cm⁻¹ originating from the vibration of C—O bond found in the ester functional group. The peak around 1400 cm⁻¹ representing the vibrations of methyl ester (CH₃—O—C(═O)—R₂) found within the biodiesel. These resulted thereby confirmed that the product synthesized was biodiesel thereby confirming that the scaling up on the production of biodiesel using the lipase and the method of the invention was successful.

### Example 17: Stability of Immobilised Enzyme after Recovery and Reuse

Tests were conducted to determine the recyclability and reuse of the immobilised enzymes using the two most active enzymes, the same being; Lip-VUT4 and Lip-VUT6 to produce biodiesel from melted animal fat for three reaction cycles. To achieve this the enzymes were then recovered from cycle 1 using magnetic bar, wash, and used to produce biodiesel in cycle 2. Then the enzymes from cycle 2 were recovered and used to produce biodiesel in cycle 3. The results of said stability tests are shown in FIG. 55.

### Example 17.1: FTIR Analysis of the Produced Biodiesel

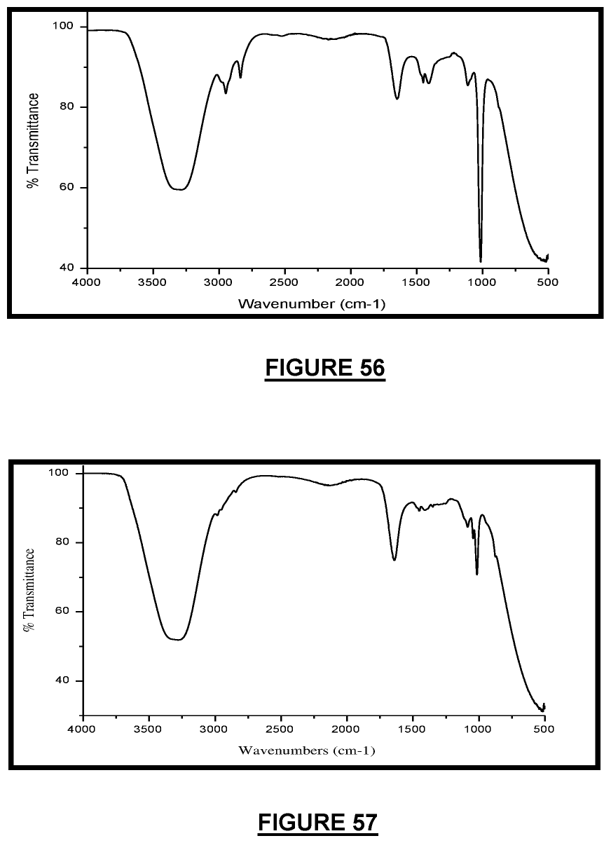

The biodiesel produced using the recycled enzyme, above, was analysed using FTIR and the results for the same are shown in FIG. 56.

The results of the FTIR analysis indicate a peak at 1010 cm⁻¹ and this peak originates from the C—O bond that is found in the ester functional group of the biodiesel. The peak around 1400 cm⁻¹ is indicative of the vibrations of methyl ester (CH₃-O—C(═O)—R₂) found within the biodiesel. As a result, it was concluded that biodiesel was successfully produced using the recycled enzyme.

### Example 17.2: Analysis of the Stability of the Recycled MNP-Enzyme Complex Using FTIR

The stability of the recycled MNP-enzyme complex was analysed using the recovered MNP-immobilized from cycle 3 and FTIR analysis to determine the presence or absence of the C—N bond between the MNP and lipase enzyme. The results of this analysis are shown in FIG. 57.

The results of the FTIR analysis showed that the MNP-enzyme complex was intact after the immobilized enzyme was recycled for the third time. This is shown by the presence of the C—N bridge around 1027 cm⁻¹ which is a result of the bond between lipase and the MNP, implying that the MNP-enzyme complex was stable post-recycling.

### Example 18: Characterisation of Lip-VUT 4

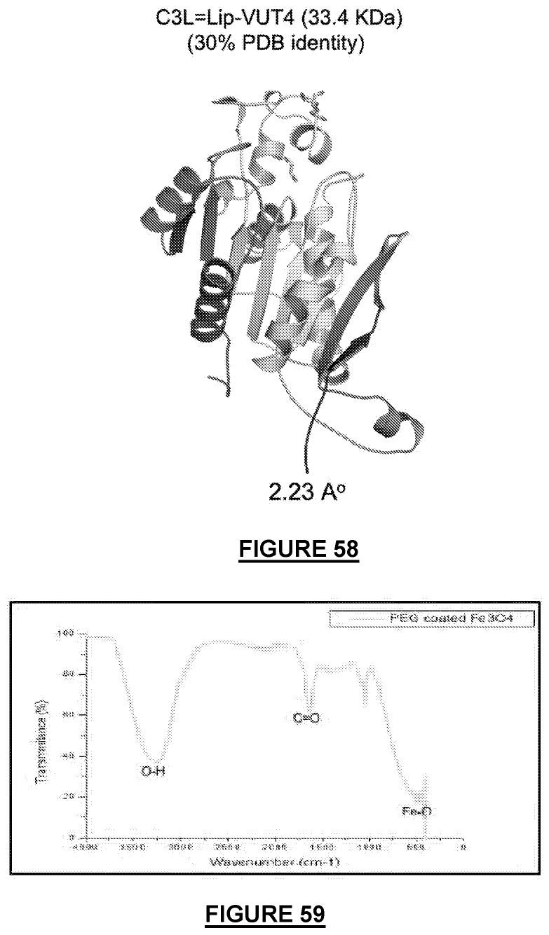

The lipase of Lip-VUT4 was found to be the most potent catalyst from the mined and characterized lipases. Hence, this enzyme was chosen to be the commercially relevant lipase and further characterised, the protein sequence (Lip-VUT4) of the same having been determined is set out in SEQ ID 1, the gene sequence of lip-vut4 in SEQ ID2 and the crystal structure in FIG. 58. When characterised further Lip-VUT4 showed further characteristics as set out in Table 4.

TABLE 4
Characteristics of lipase Lip-VUT4

| | | | PDB | | | Ext | Abs | | Optimum |
|---|---|---|---|---|---|---|---|---|---|
| | PDB | Length | identity | | MW | (w/tag;1/M*cm; | (w/tag;1g/l; | Optimum | temperature |
| Name | code | (aa) | (%) | pl | (Dalton) | No Cystine) | No Cystine) | pH | (° C.) |
| Lipase | 6NKF | 298 | 30* | 5,713 | 33366 | 46870 | 1,405 | 11 | 80 |
| Lip- | | | | | | | | | |
| VUT4 | | | | | | | | | |

*30% PDB identity is indicative of the presumptive novelty of lipase Lip-VUT4

### Example 19: Validation of the Commercial Viability of the Method for Producing Biodiesel in Accordance with the Invention at Laboratory Scale

Given the possible commercial promise shown by Lip-VUT4 the performance of the same for the conversion of larger volumes of waste oils was assessed.

### Example 19.1: FTIR-Based Analysis of the PEG-Coated Iron Magnetic Nanoparticles (Fe₃O₄) and the Immobilized Enzyme

The iron oxide nanoparticles were synthesized via the co-precipitation method using iron chloride salts and sodium hydroxide. The coating was accomplished with polyethylene glycol (PEG). The coated magnetic nanoparticles were analysed using FTIR and the result of this analysis is shown in FIG. 59.

From the FTIR spectrum peaks at the fingerprint region around 500 cm⁻¹ are seen, which represents the Fe—O bond, and which confirms that the MNP were intact.

The integrity of polyethylene glycol (PEG) was also analysed. PEG is known to have numerous OH groups, which are visible around 3000 cm⁻¹ and 3500 cm⁻¹. The presence of these peaks therefore showing that the coating material was also intact.

During the following phase FTIR was used to confirm the presence of a bond between the MNP and the PEG. The spectrum, in this instance, showed the presence of the C═O bond at 1674 cm⁻¹, which is indicative of bonds between Fe₃O₄ and PEG, which confirmed that the MNP was successfully coated with PEG. Said successful coating enabled follow up experimentation to immobilise the lipase (Lip-VUT4) on to the coated-MNP and to analyse the same using FTIR.

### Example 19.2: The FTIR-Based Analysis of the Immobilized Lipase a (Lip-VUT4)

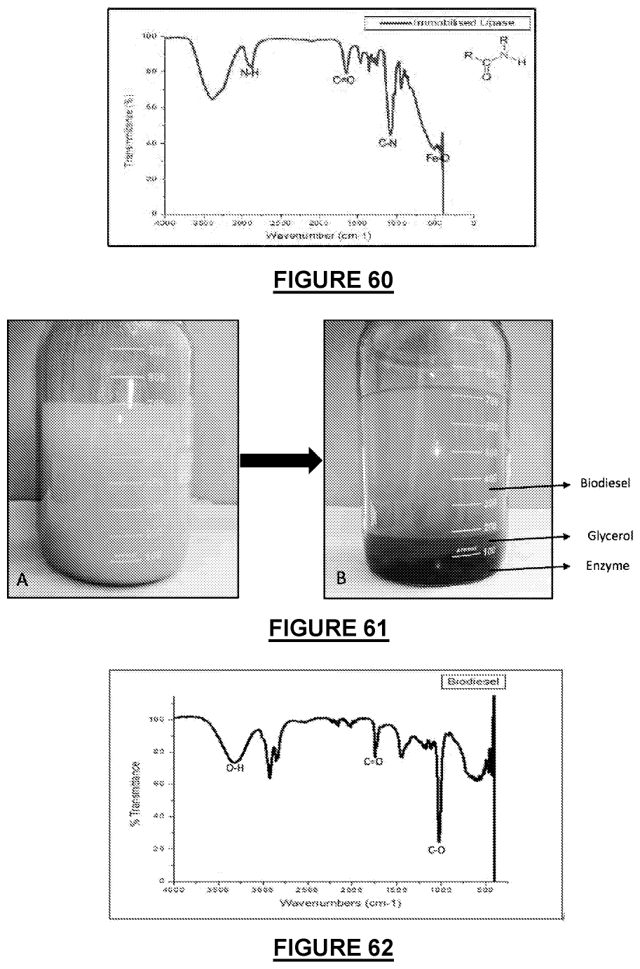

As shown above enzymes are generally immobilised on solid or mobile support to enhance their stability and improve recovery. In this instance lipase Lip-VUT4 was immobilised on PEG-coated magnetic nanoparticles. After the immobilisation step, the solution was not dried so as not tamper with the activity of the enzyme. This solution was then analysed using FTIR and the results of the same are shown in FIG. 60.

The FTIR spectrum of the MNP-PEG-Lipase complex showed that the C═O stretch of the amide is seen at 1674 cm⁻¹ that typically appears in the 1680 to 1630 range, which was a result of the bonds between Fe₃O₄ and PEG, confirming that the PEG-coated MNP was intact. Further, a C—N stretch was observed at 1240 cm⁻¹, representing the bond between nitrogen from lipase and the carbon of the PEG-coated nanoparticle.

The spectrum analysis thereby confirming that the high-performing Lip-VUT4 was successfully immobilized on the PEG-coated MNP and the same could then be used in further experimental examples.

### Example 19.3: Enzymatic Synthesis of Biodiesel (500 ml)

The reaction to produce biodiesel in accordance with the invention was optimized for a volume of 500 ml. The waste animal fat was melted and stabilized at 40° C. for 30 minutes and then filtered to remove any traces of contaminant. The ratio of fat to methanol in this instance was 1:3.

To prevent a reverse reaction from taking place, 50 ml excess methanol was added to the reaction mixture and the reaction was initiated by adding the immobilized lipase and incubating at 40° C. for 4 hours. The reaction was terminated at the end of the 4 hours by removing the enzyme from the product and the remaining mixture was heated to 150° C. to denature any traces of the enzyme.

As shown in FIG. 61 the product contained layers of biodiesel, glycerol, and immobilized enzyme.

### Example 19.4: The FTIR-Based Analysis of the Synthesized Biodiesel

FTIR spectroscopic analysis was performed to monitor the progress of the transesterification reaction of animal fat to biodiesel and the results of the same are shown in FIG. 62.

The peak at 1050 cm⁻¹ originates from the vibration of C—O bond which is found in the ester functional group. The peak around 1400 cm⁻¹ represents the vibrations of methyl ester (CH₃—O—C(═O)—R₂) found within the biodiesel. This confirmed that the product synthesized was biodiesel given the ester functional group and 0.5 L of the same was successfully produced using the methodology of the invention and the lipase.

### Example 19.5: Determining the Activity of Recovered Enzyme after Each Cycle

The activity of the MNP-immobilized enzyme was assessed after each cycle. The activity being considered at the completion of a reaction but before the commencement of the next reaction.

To assess activity a lipase activity assay was performed using, 2.5 ml of 0.1 M Tris-HCl buffer, pH 8, and 1 ml of 420 μM p-nitrophenyl palmitate substrate solution in a 15 ml test tube. One extra tube was prepared to act as a control using 1 ml of water as the control whilst adding 0.5 ml lipase solution to the remaining test tube to initiate a reaction.

The reaction was stopped after 15 minutes by placing the test tube in ice and centrifuging the sample to separate the enzyme out. Absorbance was recorded at 410 nm using a UV/VIS spectrophotometer.

Accordingly, Cycle 0 represents the activity of the enzyme before being used in the reaction to produce biodiesel. While Cycle 1 represents the activity of the recovered enzyme after the first reaction.

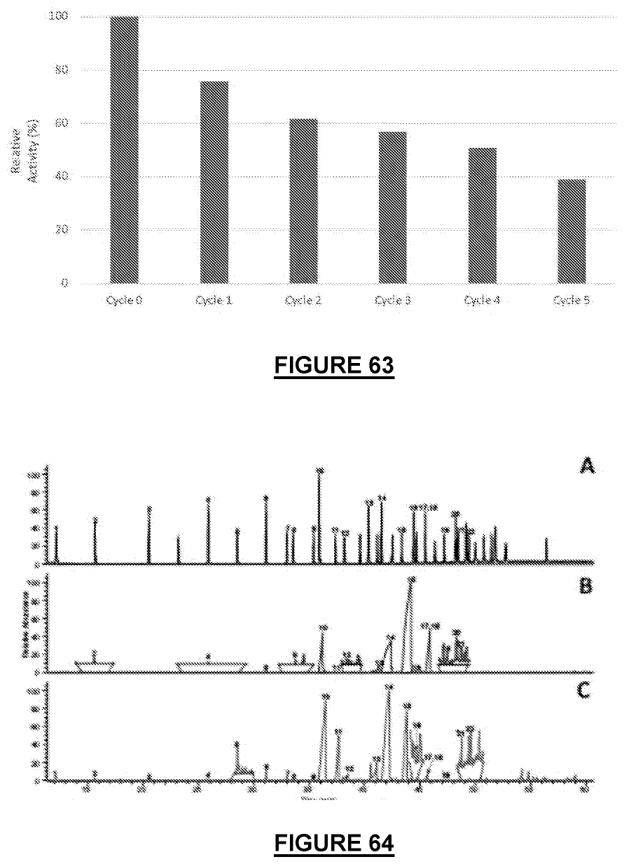

The results, as shown in FIG. 63, showed that immobilized enzyme managed to maintain more than 76% and 50% its activity after the first and fourth cycles, respectively, meaning that the same was considered to show high commercial potential for use in the production of biodiesel.

### Example 19.6: Transesterification of Waste Animal Fat

Waste animal fat was used as the substrate for producing biodiesel. The waste animal fat was filtered to remove contaminants by placing it in warm water bath for 30 minutes at 45° C. The transesterification process was performed using methanol, animal fat and MNP-immobilised enzyme as a catalyst. Excess methanol was added to the reaction to prevent the reverse reaction from taking place. The reaction was carried out at 40° C. After the reaction was complete, the enzyme was recovered and washed with 10% ethanol to remove oil and/or glycerol. The product was centrifuged to separate the biodiesel from excess methanol and glycerol.

The produced biodiesel was further analysed using Gas Chromatography Mass Spectrometry (GC-MS).

### Sample Preparation for GC-MS

A mixture of chloroform and methanol was added to ca. 2 g sample at the ratio of 2:1, respectively. The sample was vortexed and sonicated at room temperature for 30 minutes. The sample was then centrifuged at 3000 rpm for 1 minute. The bottom layer (chloroform) was dried completely with a gentle stream of nitrogen and reconstituted and vortexed with 100 μl of methyl tert-butyl ether (MTBE) and 30 μl of trimethyl sulfonium hydroxide (TMSH). One pl of the derivatized sample was injected in a 5:1 split ratio onto the GC-MS.

### Chromatographic Separation

The chromatographic separation was performed on a gas chromatograph (6890N, Agilent technologies network) coupled to an Agilent technologies inert XL EI/CI Mass Selective Detector (MSD) (5975, Agilent Technologies Inc., Palo Alto, CA). The GC-MS system was coupled to a CTC Analytics PAL autosampler. The separation of the FAMEs was performed on a polar RT-2560 (100 m, 0.25 mm ID, 0.20 μm film thickness) (Restek, USA) capillary column. Helium was used as the carrier gas at a flow rate of 1.2 ml/min. The injector temperature was maintained at 240° C. One micro litter of the sample was injected in a 5:1 split ratio. The oven temperature was programmed as 100° C. for 4 min, ramped to 240° C. at a rate of 3° C./min and held for 10 minutes.

The mass spectrometer detector (MSD) was operated in scan mode and the source and quad temperatures were maintained at 240° C. and 150° C., respectively. The transfer line temperature was maintained at 250° 5C. The mass spectrometer was operated under electron impact (EI) mode at ionization energy of 70 eV, scanning from 40 to 650 m/z. The results of the analysis are shown in FIG. 64 and in tables 5 and 6.

TABLE 5
Fatty Acid Methyl Esters (FAMES) results (concentrations)
of samples BD 100 VG and BD 100 WC in ppm (mg/l)

[symbol]

| | | BD 100 VG | BD 100 WC |
|---|---|---|---|
| [symbol] | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| | [symbol] | [symbol] | [symbol] |
| | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| | [symbol] | [symbol] | [symbol] |
| | [symbol] | [symbol] | [symbol] |
| [symbol] | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |

[symbol]

indicates data missing or illegible when filed

TABLE 6
Fatty Acid Methyl Esters (FAMES) results (concentrations) of samples
BD 100 VG and BD 100 WC in ug/g and mg/g

| | | Sample [symbol] | | | | Sample [symbol] | |
|---|---|---|---|---|---|---|---|
| | | [symbol] | [symbol] | | | [symbol] | [symbol] |
| [symbol] | [symbol] | [symbol] | [symbol] | [symbol] | [symbol] | [symbol] | [symbol] |
| | [symbol] | [symbol] | [symbol] | | [symbol] | [symbol] | [symbol] |
| | [symbol] | [symbol] | [symbol] | | [symbol] | [symbol] | [symbol] |
| | [symbol] | [symbol] | [symbol] | | [symbol] | [symbol] | [symbol] |
| | [symbol] | [symbol] | [symbol] | | [symbol] | [symbol] | [symbol] |
| | [symbol] | [symbol] | [symbol] | | [symbol] | [symbol] | [symbol] |
| | [symbol] | [symbol] | [symbol] | | [symbol] | [symbol] | [symbol] |
| | [symbol] | [symbol] | [symbol] | | [symbol] | [symbol] | [symbol] |
| | [symbol] | [symbol] | [symbol] | | [symbol] | [symbol] | [symbol] |
| | [symbol] | [symbol] | [symbol] | | [symbol] | [symbol] | [symbol] |
| | [symbol] | [symbol] | [symbol] | | [symbol] | [symbol] | [symbol] |
| | [symbol] | [symbol] | [symbol] | | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | | [symbol] | [symbol] | [symbol] |
| | [symbol] | [symbol] | [symbol] | | [symbol] | [symbol] | [symbol] |
| | [symbol] | [symbol] | [symbol] | | [symbol] | [symbol] | [symbol] |
| | [symbol] | [symbol] | [symbol] | | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | | [symbol] | [symbol] | [symbol] |
| | [symbol] | [symbol] | [symbol] | | [symbol] | [symbol] | [symbol] |
| | [symbol] | [symbol] | [symbol] | | [symbol] | [symbol] | [symbol] |
| | [symbol] | [symbol] | [symbol] | | [symbol] | [symbol] | [symbol] |
| | [symbol] | [symbol] | [symbol] | | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | | [symbol] | [symbol] | [symbol] |
| | [symbol] | [symbol] | [symbol] | | [symbol] | [symbol] | [symbol] |
| | [symbol] | [symbol] | [symbol] | | [symbol] | [symbol] | [symbol] |
| | [symbol] | [symbol] | [symbol] | | [symbol] | [symbol] | [symbol] |
| | [symbol] | [symbol] | [symbol] | | [symbol] | [symbol] | [symbol] |
| | [symbol] | [symbol] | [symbol] | | [symbol] | [symbol] | [symbol] |
| | [symbol] | [symbol] | [symbol] | | [symbol] | [symbol] | [symbol] |

[symbol]

indicates data missing or illegible when filed

### GC-MS Analysis Result

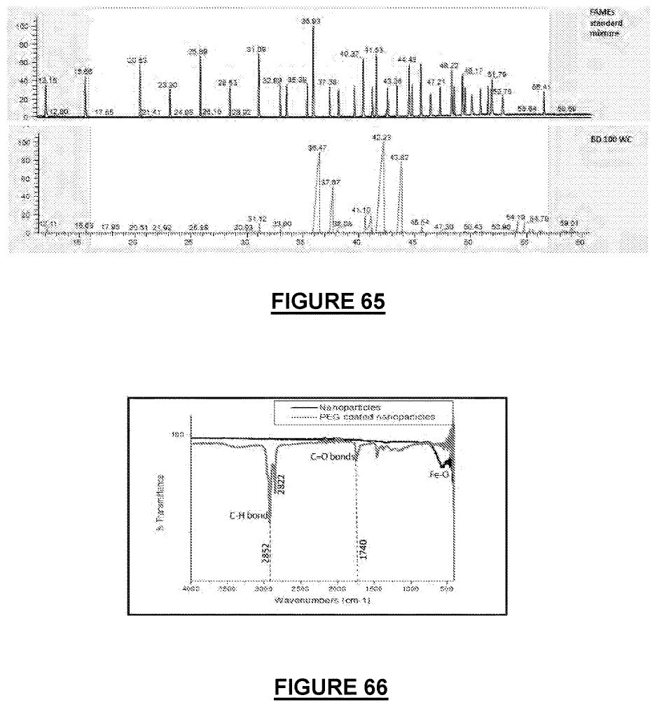

Purified waste animal fat was used as substrates for methyl ester production with MNP-immobilised lipase (Lip-VUT4). The fatty acid methyl ester contents of the produced biodiesel were analysed using the Agilent Technologies GC-MS systems. The gas chromatograms observed in FIG. 65 illustrate the retention time of the FAMEs present in the biodiesel against the FAMEs standard mixture.

It was further shown that the biodiesel consisted of more monounsaturated fatty acids. The total fatty acid content was 58.74% (table 7).

TABLE 7
Fatty acid methyl ester composition of the synthesized
biodiesel according to GC-MS analysis.

| | | mg/g |
|---|---|---|
| Saturated Fatty Acid Methyl Esters | | |
| Caproic Acid Methyl Ester | C6:0 | 0.274 |
| Caprylic Acid Methyl Ester | C8:0 | 0.197 |
| Capric Acid Methyl Ester | C10:0 | 0.036 |
| Lauric Acid Methyl Ester | C12:0 | 0.029 |
| Tridecanoic Acid Methyl Ester | C13:0 | 0.006 |
| Myristic Acid Methyl Ester | C14:0 | 0.456 |
| Pentadecanoic Acid Methyl Ester | C15:0 | 0.047 |
| Palmitic Acid Methyl Ester | C16:0 | 15.984 |
| heptadecanoic Acid Methyl Ester | C17:0 | 0.015 |
| Steric Acid Methyl Ester | C18:0 | 2.092 |
| Arachidic Acid Methyl Ester | C20:0 | 0.02 |
| Behenic Acid Methyl Ester | C22:0 | not detected |
| Monounsaturated Fatty Acids Methyl Esters | | |
| Myristic Acid Methyl Ester | C14:1 | 0.176 |
| Cis-10-Pentadecenoic Acid Methyl Ester | C15:1 | 0.027 |
| Palmitoleic Acid Methyl Ester | C16:1 | 4.946 |
| Oleic Acid Methyl Ester | C18:1 | 22.561 |
| Cis-11-Eicoseinoic Acid Methyl Ester | C20:1 | 0.227 |
| Polyunsaturated Fatty Acid Methyl Esters | | |
| Linoleic Acid Methyl Ester | C18:2N6 | 11.290 |
| Linolenic Acid Methyl Ester | C18:3N3 | 0.227 |
| Eicosatrienoic Acid Methyl Ester | C20:2N6 | 0.042 |
| Arachidonic Acid Methyl Ester | C20:4N6 | 0.042 |
| Total FAMEs content | | 58.74% |

The immobilized lipase demonstrated high capacity in catalyzing transesterification reaction using waste animal fat as substrate obtaining FAME conversion of 58.74±0.69% in a 4 h reaction, meaning that biodiesel was successfully synthesized using animal fat as a substrate.

### Example 19.7: Synthesis Coating and FTIR-Analysis of Magnetic Nanoparticles

Magnetic nanoparticles were synthesized, coated with polyethylene glycol (PEG), and analysed using FTIR.

### Synthesis and Analysis of Magnetic Nanoparticle

The iron oxide magnetic nanoparticles were prepared by dissolving 14.33 g of iron II chloride and 39.2 g of iron III chloride in 1 litre of boiling water. The mixture was allowed to cool, and sodium hydroxide was added drop-wise to precipitate the nanoparticles. The pH of the solution was adjusted to 11. A black precipitate was formed at the end of the reaction, which is an indication of the successful synthesis of the MNP. The nanoparticles were then washed with water and ethanol. The liquid was discarded, and the nanoparticles were air dried. The FTIR spectra of the synthesized magnetic nanoparticles (in black) and the PEG-coated magnetic nanoparticles (in red) are shown in FIG. 65.

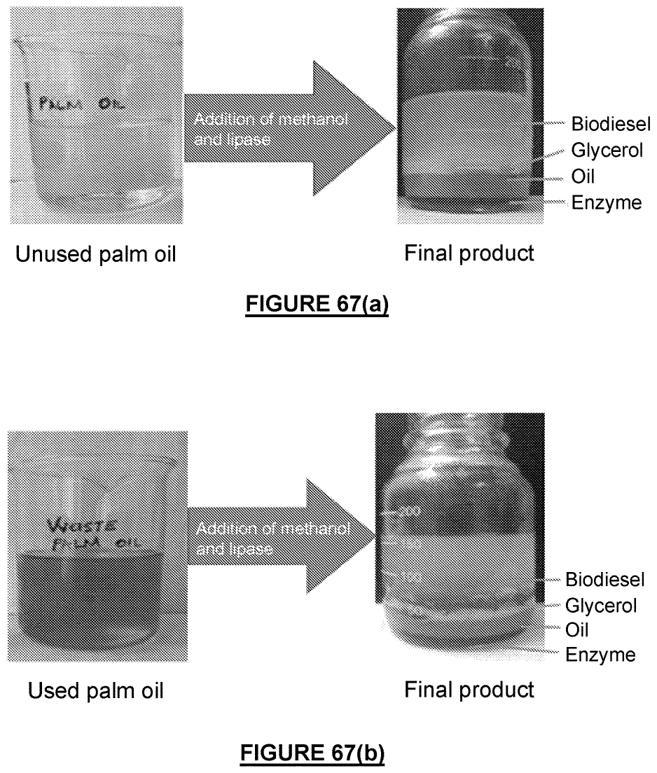

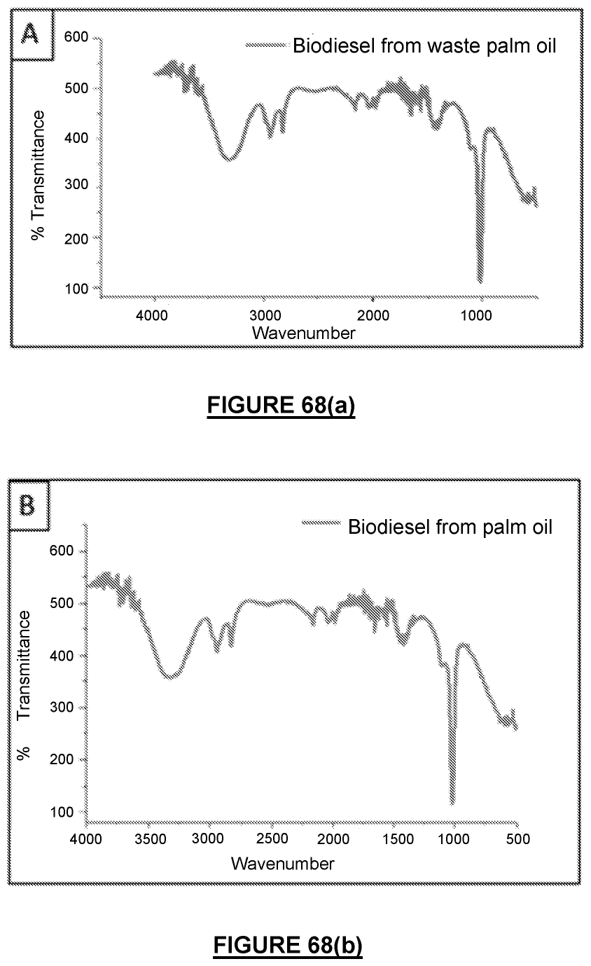

As a result, the peaks appeared around 500 cm, which was an indication of the Fe—O bond present in the MNPs (FIG. 66). This indicates that the iron oxide magnetic nanoparticles with ferrimagnetic properties were successfully synthesized.

Coating of Magnetic Nanoparticles with Polyethylene Glycol (PEG)

The synthesized magnetic nanoparticles were dispersed in ethanol. This allows easy coupling of polyethylene glycol and magnetic nanoparticles. Polyethylene glycol was added to the suspension and incubated at 30° C. for coating, on a shaking platform and FTIR analysed.

Accordingly, intense absorptions of the light were observed at 590, 1740, 2822, and 3852 cm⁻¹ in the spectrum for the nanoparticles coated with PEG. The FTIR band around 1740 cm⁻¹ is characteristic of the C═O stretching mode of the bond between PEG and the nanoparticles. The aliphatic C—H stretching at 2822 and 2852 cm⁻¹ was due to the C—H vibration of PEG.

The MNPs were successfully synthesized and coated with polyethylene glycol (PEG), and the integrity of the PEG-coated MNPs using FTIR was confirmed.

### Example 20.1: Production of Biodiesel from Waste Cooking Oil and Waste Palm Oil Using Immobilised Lipase

The cooking oil was stabilized by placing it in a water bath for 30 minutes at 50° C. Following stabilization 40 ml of oil and 120 ml of methanol was added and mixed using a magnetic stirrer.

The immobilised enzyme was thawed at room temperature and resuspended before 15 ml of the same was added to the oil mixture for transesterification. The reaction mixture was then incubated in a shaker incubator for 18 hours at 40° C., continuously stirring at 200 rpm.

After incubation the products were transferred to a separatory funnel and the mixture was allowed to settle for 15 minutes. Upon separation two layers were visible as is shown in FIG. 67. The immobilised enzyme (black layer) was removed and added to a centrifuge tube.

Enzyme was stored at −4° C. for further washing and removal of by-products. The top layer of biodiesel was transferred to a 50 ml centrifuge tube and centrifuged at 15 000 rpm for 15 minutes, temperature of 4-9° C. The supernatant was added to a fresh centrifuge tube and placed the tube in a beaker with boiling water for 5 minutes to denature any present enzyme. The biodiesel was stored for FTIR analysis.

### Example 20.2: FTIR Analysis of the Produced Biodiesel

The generated biodiesel was examined using FTIR. With two different substrates, two distinct reactions were carried out. About 99% of the spectra of the biodiesels from used cooking oil were comparable as is seen in FIG. 68. There was a peak at 1010 cm⁻¹ that was caused by the C—O bonds in the ester functional group of fatty acid methyl esters (FAMEs). The biodiesel's methyl ester (CH₃—O—C(═O)—R₂) vibrations can be seen as a peak at 1400 cm⁻¹. It would be clear to a person skilled in the art that biodiesel was successfully produced using the abovementioned methodology using the immobilised lipase (Lip-VUT4) of the invention on a larger scale 500 ml volume.

### REFERENCES

1. Biodiesel solutions; Biodiesel News (2018); Preparation of Biodiesel by Enzyme Method. Preparation Of Biodiesel by Enzyme Method—Biodiesel Solutions—Biodiesel Machinery Equipment Projects. (biodieselproject.com)
2. Cognis IP Management GmBH CA2595007—Compositions which can be used as biofuel
3. Hycagen Ltd et al WO2011104528—Biodiesel compositions
4. Jilse Sebastian, Chandrasekharan Muraleedharan & Arockiasamy Santhiagu I An-Ping Zeng (Reviewing Editor) (2016) A comparative study between chemical and enzymatic transesterification of high free fatty acid contained rubber seed oil for biodiesel production, Cogent Engineering, 3:1, DOI: 10.1080/23311916.2016.1178370
5. Maria Manuela Camino Feltes, Debora de Oliveira, Jorge Luiz Nonow and Jose Vladimir de Oliveira (2011). An overview of enzyme-catalysed reactions and alternative feedstock for biodiesel production. IntechOpen
6. Nielsen Per Munk, Novozymes A/S and Holm Hans C. US20140017741-Esterification Process
7. Novozymes A/S and N. V. Desmet Ballestra Group S. A.US20100047884-Producing Esters of Fatty Acid and C1-C3 Alkyl Alcohols
8. Silvia Daniela Romano and Patricio Anibal Sorichetti (2011) Dielectric Spectroscopy in Biodiesel Production and Characterization, Chapter 2.

1. An immobilised lipase having protein SEQ ID1, said immobilised lipase being immobilised on an adsorbent substrate.

2. The immobilised lipase, as claimed in claim 1, where the adsorbent substrate is a nanoparticle.

3. The immobilised lipase, as claimed in claim 2, wherein the nanoparticle is an organic nanoparticle, alternatively an inorganic nanoparticle.

4. The immobilised lipase, as claimed in claim 3, wherein the inorganic nanoparticle is selected from the group comprising: mesoporous silica, organic polymers, protein-coated microcrystals, and MNPs.

5. The immobilised lipase, as claimed in claim 4, wherein the MNPs are coated-iron MNPs.

6. The immobilised lipase, as claimed in claim 5, wherein the coated-iron MNPs are coated with silicon dioxide (SiO₂), alternatively glutamic acid, further alternatively Polyethylene Glycol (PEG), most preferably with PEG, and are recoverable, in use, from a reaction product using a magnetically active material.

7. A lipase isolated from the goat gut rumen metagenome having protein SEQ ID 1.

8. The lipase, as claimed in claim 7, wherein gene SEQ ID 2 encodes protein SEQ ID 1.

9. The lipase, as claimed in claim 7, wherein the lipase is about 33.4 KDa.

10. The lipase, as claimed in claim 7, wherein the lipase has optimum catalysis conditions at pH 11 and 80° C.

11. The lipase, as claimed in claim 7, having a pl of 5,713.

12. A method of converting a waste oil to biodiesel, the method comprising utilizing a lipase, as claimed in claim 1 or claim 7, to catalyse the conversion of said waste oil to biodiesel in the presence of a short-chain alcohol in accordance with the following general reaction: 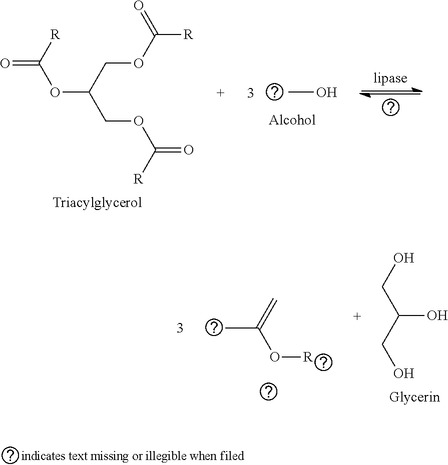
wherein R and R1 represent an alkyl chain of different lengths and/or saturation degrees.

13. The method of converting a waste oil to biodiesel, as claimed in claim 0, wherein the triacylglycerol is selected from the group comprising: saturated, monounsaturated, and polyunsaturated triacylglycerols.

14. The method of converting a waste oil to biodiesel, as claimed in claim 0, wherein the waste oil is a plant-based alternatively, an animal-based waste oil.

15. The method of converting a waste oil to biodiesel, as claimed in claim0, wherein the short-chain alcohol is selected from the group comprising: methanol, ethanol, butanol, propanol and amylic alcohol, most preferably methanol.

16. The method of converting a waste oil to biodiesel, as claimed in claim0, wherein the lipase is a free enzyme, alternatively an immobilised lipase.

17. The method of converting a waste oil to biodiesel, as claimed in any one of claims 12 to 16, wherein the conversion of the waste oil feedstock to biodiesel takes place in a batch process, said process being conducted in a stirred-tank bioreactor, membrane bioreactor, fluidized bed reactor or packed-bed bioreactor.

18. The lipase, as claimed in claim 1 or claim 7, for use in a method of converting a waste oil to biodiesel, said method comprising contacting a waste oil feedstock with said lipase and allowing the same to catalyse the conversion of said waste oil to biodiesel in accordance with the reaction of claim 12.

19. A nanoparticle to which a lipase, as claimed in claimed in any one of claim 7 to 11, is immobilised.

20. A biodiesel product produced in accordance with the method of converting a waste oil to biodiesel as claimed in any one of claims 0 to 17.

21. An immobilised lipase, a lipase, a method of converting a waste oil to biodiesel, a nanoparticle, and a biodiesel product, substantially as described, and exemplified herein with reference to the accompanying examples.
